(19) United States
(12) Patent Application Publication
(10) Pub. No.: US 2019/0154679 A1
(43) Pub. Date: May 23, 2019

(54) APPARATUSES AND METHODS FOR CELL AND TISSUE ASSAYS AND AGENT DELIVERY

(71) Applicants: Massachusetts Institute of Technology, Cambridge, MA (US); Beth Israel Deaconess Medical Center, Boston, MA (US)

(72) Inventors: Patrick S. Doyle, Sudbury, MA (US); Augusto M. Tentori, Somerville, MA (US); Maxwell Benjamin Nagarajan, Prior Lake, MN (US); Jae Jung Kim, Cambridge, MA (US); Wen Cai Zhang, Brookline, MA (US); Frank J. Slack, Waban, MA (US)

(73) Assignees: Massachusetts Institute of Technology, Cambridge, MA (US); Beth Israel Deaconess Medical Center, Boston, MA (US)

(21) Appl. No.: 16/191,368

(22) Filed: Nov. 14, 2018

Related U.S. Application Data
(60) Provisional application No. 62/585,771, filed on Nov. 14, 2017.

(51) Int. Cl. G01N 33/543; B01L 3/00

(52) U.S. Cl. CPC G01N 33/54366; B01L 3/502715; B01L 2300/0893; B01L 2300/0819; B01L 2300/0829

(57) ABSTRACT

Apparatuses and methods for cell and tissue assays and agent delivery are generally described.

### RELATED APPLICATIONS

This application claims priority under 35 U.S.C. § 119(e) to U.S. Provisional Patent Application Ser. No. 62/585,771, filed Nov. 14, 2017, and entitled “Bioassays from Tissue Sections and Cells Using Functionalized Hydrogels in Isolated Microwell Arrays,” which is incorporated herein by reference in its entirety for all purposes.

### FIELD

Apparatuses and methods for cell and tissue assays and agent delivery are generally described.

### BACKGROUND

Cell and tissue assays of analytes (e.g., cells, molecules) have been used to diagnose disease states. These assays may have low throughput, low sensitivity, and/or a limited ability to multiplex for the detection of multiple analytes simultaneously, which limits their utility in a clinical setting.

Delivery of agents at high throughput to many containers (e.g., wells) simultaneously presents a challenge in cell and tissue assays and in other applications in medicine and engineering.

Accordingly, improved apparatuses and methods for assaying cells and/or tissues and for delivery of agents are needed.

### SUMMARY

Apparatuses and methods for cell and tissue assays and agent delivery are provided. The subject matter of the present invention involves, in some cases, interrelated products, alternative solutions to a particular problem, and/or a plurality of different uses of one or more systems and/or articles.

In one aspect, an apparatus is provided. In some embodiments, an apparatus comprises: a first microwell array, wherein each microwell in the first microwell array comprises: a first post in the microwell; wherein the first post comprises a first probe configured to detect a first analyte.

In another aspect, a method is provided. In some embodiments, a method is a method of assaying a first analyte in a biological sample. In some embodiments, a method comprises: exposing a biological sample to a first probe on a first post in a first microwell, wherein the first probe is configured to detect a first analyte.

In some embodiments, a method of assaying an analyte in a tissue sample (e.g., tissue section) is provided. In some embodiments, a method comprises positioning separate probe articles proximate to separate areas of a surface of the tissue sample, wherein at least some of the separate probe articles are configured to detect a first analyte.

In some embodiments, a method of delivering an agent is provided. In some embodiments, the method comprises wetting a first substrate with a liquid comprising the agent; and positioning a surface of the first substrate proximate to a surface of a first microwell array such that the contents of each microwell in the first microwell array are physically separated from one another.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

In the figures:

FIG. 1 is a side cross-sectional view schematic of an apparatus 100 for cell and/or tissue assays, according to some illustrative embodiments;

FIG. 2 depicts a side cross-sectional view schematic of a microwell 104 (left), and top cross-sectional view schematics of two alternative configurations 110 and 112 (right) of a microwell, according to some illustrative embodiments;

FIG. 3 depicts a side cross-sectional view schematic of a first multiplexed microwell 120, according to some illustrative embodiments;

FIG. 4 depicts a side cross-sectional view schematic of an apparatus comprising a first microwell 104 and a substrate 202, according to some illustrative embodiments;

FIG. 5A is a non-limiting schematic of an apparatus for a cell assay and/or tissue assay, in accordance with certain embodiments;

FIG. 5B is a non-limiting schematic of an assay method for miRNA using the apparatus of FIG. 5A, in accordance with certain embodiments;

FIG. 6A is a schematic of a method of assaying miRNAs in microwells, according to certain embodiments;

FIG. 6B is a composite fluorescence and brightfield image of an miR-21 assay using sealed microwells, according to certain embodiments;

FIG. 6C shows brightfield images of Calu-6 cells after settling (top right) and fluorescence images after miR-21 assay method (bottom right) in comparison with no cells (left), according to certain embodiments;

FIG. 6D shows plots of average (top) and individual (bottom) net fluorescence from posts in microwells in an miR-21 assay, according to certain embodiments;

FIG. 7A is a fluorescence micrograph of an enzymatic reaction (between an analyte and a probe) in microwells 1 hr after sealing with a substrate comprising smaller microwells, according to certain embodiments;

FIG. 7B is a fluorescence plot of the enzymatic reaction of FIG. 7A, according to certain embodiments;

FIG. 7C shows brightfield (top) and fluorescence (bottom) images of microposts of different sizes after a miR-21 assay in sealed microwells, according to certain embodiments;

FIG. 7D shows net mean fluorescence intensity plots of microposts of different sizes after the miR-21 assay of FIG. 7C in sealed microwells, according to certain embodiments;

FIG. 8A shows brightfield (top) and fluorescence (bottom) images of differen numbers of microposts after a miR-21 assay in sealed microwells, according to certain embodiments;

FIG. 8B shows net mean fluorescence intensity plots of microposts of different sizes and/or numbers after the miR-21 assay of FIG. 7C or FIG. 8A in sealed microwells, according to certain embodiments;

FIG. 9A is a schematic of an assay for miRNA (e.g., miR-21) similar to FIG. 6A, in accordance with certain embodiments;

FIG. 9B shows brightfield images (top) and fluorescence micrographs (bottom) of hydrogel posts in microwells after assays done with different miR-21 “mass” (attomoles per well), in accordance with certain embodiments;

FIG. 9C shows plots of net fluorescence from posts having miR-21 probes vs. mass per well of miR-21, in accordance with certain embodiments;

FIG. 10A is a schematic of an miRNA (e.g., miR-21) tissue section assay using a microwell array with posts, in accordance with certain embodiments;

FIG. 10B shows brightfield and fluorescence composite images of microwells after a miRNA (e.g., miR-21) assay of FIG. 10A, in accordance with certain embodiments;

FIG. 10C shows column graphs showing fluorescence intensity signal from wells in a microwell array, in an miR-21 tissue section assay as in FIG. 10A, according to certain embodiments;

FIG. 10D shows a heat map of fluorescence signal from a tissue section as analyzed in a microwell array in an miR-21 assay as in FIG. 10A, in accordance with certain embodiments;

FIG. 11A shows an experimental setup schematic of multiplex miRNA profiling from fixed tissue sections, in accordance with certain embodiments;

FIG. 11B shows brightfield (top) and fluorescence (bottom) images of wells following the miRNA multiplex assay of FIG. 11A with fixed tissue with paraffin removed, in accordance with certain embodiments;

FIG. 11C shows brightfield (top) and fluorescence (bottom) images of wells following the miRNA multiplex assay of FIG. 11A with fixed tissue without paraffin removed, in accordance with certain embodiments;

FIG. 12 shows a schematic of fabrication of different posts within a single well, in accordance with certain embodiments;

FIG. 13A is a schematic of a method of gel post fabrication, in accordance with certain embodiments;

FIG. 13B shows brightfield and fluorescence composite images of biotinylated and blank gel posts, fabricated in alternating steps, after streptavidin, r-phycoerythrin conjugate (SA-PE) binding, in accordance with certain embodiments;

FIG. 13C shows plots of mean fluorescence intensities for each post (top) and mean values for biotinylated and blank posts (bottom), in accordance with certain embodiments;

FIG. 14 is a schematic of an assay protocol for a nucleic acid analyte, using a microwell comprising a post having a probe for the analyte, in accordance with certain embodiments;

FIG. 15 shows bright field images of microwell arrays, in accordance with certain embodiments;

FIG. 16A shows brightfield (top) and fluorescence (bottom) micrographs following an SA-PE binding assay, in accordance with certain embodiments;

FIG. 16B shows box plots showing the distribution of mean fluorescence of posts in each condition of FIG. 16A, in accordance with certain embodiments;

FIG. 16C shows a plot showing mean post fluorescence vs. loaded mass for each well of FIG. 16A, in accordance with certain embodiments;

FIG. 17A shows brightfield (top) and fluorescence (bottom) micrographs following multiplex miRNA hybridization assay in sealed wells, in accordance with certain embodiments;

FIG. 17B shows plots showing net mean fluorescence for different miRNAs as a function of loaded mass per well in the multiplexed miRNA assays of FIG. 17A, in accordance with certain embodiments;

FIG. 18A shows brightfield images after cell settling (top), and following assay (middle); and fluorescence micrograph (bottom) of representative well following multiplex miRNA assays from Calu-6 cells in a well array, in accordance with certain embodiments; and

FIG. 18B is a plot of net mean fluorescence for miRNA targets in the multiplex miRNA assays of FIG. 18A, in accordance with certain embodiments.

### DETAILED DESCRIPTION

The present disclosure is directed to apparatuses and methods for cell and tissue assays and agent delivery. In some embodiments, the apparatus includes a microwell array, where each microwell contains a probe article (e.g., post) having a probe (e.g., comprising a nucleic acid) configured to detect an analyte (e.g., microRNA (miRNA)). The microwell may be configured to hold a small volume (e.g., between or equal to 0.1 nL and 50 microliters, between or equal to 1 nL and 5 nL). In certain embodiments, cells can be concentrated into this small volume (e.g., by settling) and/or cell contents from proximate tissue can diffuse through the small volume in a relatively short timescale (e.g., between or equal to 1 minute and 20 minutes). The probe article (e.g., post) may have relatively small dimensions (e.g., a diameter of, e.g., between or equal to 1 micron and 80 microns; a cross-sectional area of, e.g., between or equal to 1 microns² and 8000 microns²). In certain embodiments, the relatively small dimension of the probe article facilitates an analyte captured by the probe on the probe article to be detected at high sensitivity (e.g., with a lower limit of detection of 0.001 amol and 0.010 amol). Some methods of assaying a biological sample (e.g., cell sample, tissue sample) disclosed herein involve capturing an analyte (e.g., from a cell and/or tissue, e.g., from a lysed cell) with a probe on a probe article (e.g., post) in a microwell. Some methods of assaying a tissue sample disclosed herein involve contacting separate probe articles (e.g., posts) to separate areas of a surface of the tissue sample (e.g., tissue section). In some embodiments, methods of assaying a biological sample involve using an apparatus described herein. Some methods of delivering an agent disclosed herein involve wetting a first substrate (e.g., comprising a microwell array) with a liquid containing the agent and then positioning a surface of the first substrate proximate (e.g., in contact with) a surface of a first microwell array such that the contents of each microwell in the first microwell array are physically separated from one another.

As used herein, the term “assay” will be understood by those of ordinary skill in the art and refers to a method of measuring the presence, quantity, and/or activity of an analyte.

In some embodiments, an apparatus advantageously allows for a microwell to be readily multiplexed such that a plurality of analytes can be assayed simultaneously for a single cell or a small number of cells (e.g., at most 100 cells, at most 10 cells). In some embodiments, the biological samples being analyzed are low in number of cells, e.g., 3D spheroids, circulating cell clusters organoids, early stage embryos, small whole organisms, and biopsies.

While certain embodiments of the current disclosure are applicable at least to microRNA (miRNA) assays, it should be understood that the current disclosure is not limited to assaying any particular type of analyte. Instead, any appropriate analyte (e.g., proteins, messenger RNA (mRNA), other nucleic acids, cytokines) may be assayed that is capable of binding to a suitable probe.

Apparatuses and methods described herein are directed to a new approach to assaying analytes (e.g., cells, biomarkers) from a biological sample (e.g., a cell sample, a tissue sample, e.g., from a human patient). These apparatuses and methods have utility for diagnosing any disease state where an analyte from a biological sample is to be analyzed in situ and/or the spatial location of the analyte in a tissue sample is an important piece of information to retain in the assay. By assaying for certain analytes in a biological sample using apparatuses and methods described herein, a clinician may be able to make inferences on the disease status of the patient. For example, an assay that reveals the presence of multi-nuclear cells may indicate to the clinician a diagnosis of a certain cancer. By assaying a biological sample for a certain analyte, a clinician may be able to determine whether or not a particular therapy is available to a particular patient. In certain embodiments, a clinician might want to know quantitatively and spatially where certain analytes (e.g., biomarkers) are present and/or being expressed in a tissue sample.

Alternative apparatuses and methods of cell and/or tissue assays involving retaining spatial location information suffer from limitations that impede their use in a clinical setting. For example, fluorescent in situ sequencing, which involves converting ribonucleic acid (RNA) into cross-linked complementary deoxyribonucleic acid (cDNA) amplicons and sequencing manually on a confocal microscope, has significantly lower sensitivity as compared with assays described in the current disclosure as applied to similar RNA analytes. This higher sensitivity of assays in the current disclosure is due in part to the small diameter of the probe articles (e.g., posts) and/or the small volume of the microwells in apparatuses described herein. As another alternative example, laser capture microdissection, which involves isolating specific cells of interest from a biological sample using a laser coupled to a microscope, is tedious and time-consuming and can produce assay results for only a small number of cells at a time (low throughput), rather than for a surface of a biological sample of any suitable size as allowed by the apparatuses and methods described herein. In some methods herein, by placing separate probe articles in close proximity to separate areas of a surface of a tissue sample, high throughput analysis of the separate areas of the tissue sample simultaneously is possible while retaining spatial information. Apparatuses and methods described herein advantageously facilitate sensitive, quantitative, and rapid assaying of cell and tissue samples while retaining spatial location information.

Apparatuses described herein can advantageously be readily multiplexed (e.g., by introducing two or more probe types in close proximity, e.g., in the same microwell, e.g., on the same probe article or separate probe articles) to facilitate detection of multiple analytes simultaneously.

In some embodiments, each microwell is physically isolated from any other microwells (e.g., in a microwell array). In some such embodiments, a microwell is advantageously configured to serve as a reaction vessel for any binding between any analytes present in the vessel and any corresponding probes configured to capture the analytes. In some embodiments, physical isolation of each microwell from any other microwell advantageously results in a lack of overlap between analyte information from one region of a biological sample (e.g., the tissue sample) and another region of the biological sample.

In some embodiments, the relatively small diameter of probe articles (e.g., posts) and/or the relatively small volume of microwells advantageously facilitate increased sensitivity of cell and/or tissue assays, such that quantification is possible for analytes that are single cells or contained in single cells, or for analytes that are a few cells (e.g., at most 10 cells) or are contained in a few cells.

Some apparatuses and methods of the present disclosure advantageously facilitate obtaining spatially resolved localized information from a tissue sample (e.g., a tissue section), by providing separate probe articles (e.g., posts) to be contacted to separate areas of a surface of the tissue sample. Some apparatuses and methods described herein facilitate high-throughput and/or multiplexed detection of analytes (e.g., miRNA) from cells in a tissue sample while preserving the spatial information of the tissue sample (e.g., tissue section), which in some cases is important for an accurate diagnosis of a disease state (e.g., cancer, neurodegenerative disease).

Cell and/or tissue assays that are conducted in bulk (e.g., in a volume of greater than 50 microliters, e.g., 100 microliters) suffer from difficulties capturing analytes (e.g., cells, molecules) with probe articles (e.g., particles), at least due to the relatively low concentration of analytes in the relatively large volume of liquid. Some apparatuses and methods of assaying cells and/or tissues described herein operate using smaller volumes (e.g., less than or equal to 50 microliters, in microwells), resulting in a higher concentration of analytes that are more easily captured by probe articles (e.g., posts) in part due to a lesser distance required to for the analytes to diffuse to reach the probes.

Cell and/or tissue assays that are conducted in bulk using particles as probe articles tend to have redundancy, resulting in an analyte signal being spread over a plurality of different particles (e.g., microparticles). By contrast, some apparatuses and/or methods described herein result in a single probe article (e.g., post) detecting an analyte for a particular cell or group of cells (e.g., in a microwell), increasing the sensitivity of the assay relative to bulk particle probe assays. In addition, by decreasing the diameter of a probe-containing post in a microwell (e.g., protruding from the base of a microwell), the sensitivity of the assay can be further increased, especially in cases where a method of measuring the quantity of analyte captured by the post involves imaging a microwell or microwell array using a photodetector facing the base of the microwell.

Cell and/or tissue assays have generally required sample preparation separate from the assay method. By contrast, some apparatuses and/or methods described herein facilitate a one pot assay that introduces an agent (e.g., a lysis buffer) for sample preparation to cells and/or a tissue sample that is/are localized with probe articles (e.g., posts, e.g., in microwells). For example, in some embodiments, a method of assaying a tissue comprises applying a liquid comprising a lysis buffer and/or a probe-analyte interaction buffer (e.g., a probe-analyte hybridization buffer) to a substrate comprising a plurality of separate probe articles that are posts protruding from the substrate, and then contacting the separate probe articles to separate areas of a surface of a tissue sample (e.g., tissue section), thereby also exposing the tissue sample to the liquid. As another example, in some embodiments, a method of assaying cells comprises: applying a liquid comprising a lysis buffer and/or a probe-analyte interaction buffer to a first substrate (e.g., comprising microwells); applying cells (e.g., in a fluid suspension) to a second substrate comprising a plurality of microwells (e.g., having a diameter and/or a spacing (e.g., a minimum spacing) between microwells that is/are larger than the diameter and/or spacing between microwells of the first substrate), each of which microwells comprises a post comprising a probe configured to detect the analyte; and positioning a surface of the first substrate proximate to a surface of the second substrate such that the contents of each microwell in the second substrate are physically separated from one another.

In certain embodiments, it may be advantageous to deliver an agent to a plurality of microwells simultaneously. By wetting a first substrate with a liquid containing the agent, and then positioning a surface of the first substrate proximate to a surface of a first microwell array, the agent can be delivered to each microwell in the microwell array simultaneously in a straightforward manner. In addition, the surface of the first substrate can be positioned proximate to the surface of the first microwell array such that the contents of each microwell are physically separated from one another, so that the agent is delivered into each microwell without introducing the possibility of mixing the contents of a microwell with the contents of an adjacent microwell.

Some embodiments of the present disclosure are directed to methods of assaying nucleic acids (e.g., microRNAs (miRNAs)). Some previous methods of assaying nucleic acids have involved in situ hybridization, which have been qualitative methods suffering from difficulties quantifying and limitations on multiplexing—on being able to assay for multiple nucleic acids (e.g., microRNAs) simultaneously. For example, methods of assaying microRNAs have been limited to assaying two or three miRNAs at the same time, and such methods might require on the order of 1000 different experiments in order to assay all of the miRNAs of interest. By contrast, apparatuses and methods described herein facilitate assaying up to 2500 or more miRNAs (e.g., human miRNAs) simultaneously. For example, an apparatus comprising a microwell array in which each microwell has a plurality of probes (e.g., on one or more probe articles (e.g., posts)), can facilitate a multiplexed assay that results in fewer experiments required to analyze a biological sample. For example, each probe article in a microwell may have a different corresponding probe configured for detecting a different miRNA, and/or each probe article in a microwell may have a plurality of different corresponding probes configured for detecting different miRNAs.

In some embodiments, an apparatus comprises a microwell. As used herein, the term “microwell” refers to a vessel having at least one dimension (e.g., diameter, height) between or equal to 1 micron and 1000 microns.

In some embodiments, a microwell has a largest lateral dimension (e.g., diameter, in embodiments where a microwell is cylindrical and has a circular cross-section) on the micron scale. In certain embodiments, this small largest lateral dimension results in smaller distances for analytes to travel to reach probes in the microwell, resulting in faster assays, as further described herein. In certain other embodiments in which an apparatus comprises a first microwell and a second microwell array, a small largest lateral dimension and/or a small distance between second microwells in the second microwell array (relative to the largest lateral dimension of the first microwell and/or the distance between first microwells in a first microwell array) facilitates physically isolating a first microwell from an external environment by positioning a surface of the first microwell proximate (e.g., in contact with) a surface of the second microwell array. In some embodiments, a microwell has a largest lateral dimension of at least 1 micron, at least 5 microns, at least 10 microns, at least 25 microns at least 100 microns, at least 200 microns, or at least 300 microns. In some embodiments, a microwell has a largest lateral dimension of at most 1000 microns, at most 500 microns, or at most 400 microns. Combinations of the above-referenced ranges are also possible (e.g., between or equal to 1 micron and 1000 microns, between or equal to 10 microns and 400 microns, between or equal to 25 microns and 400 microns, between or equal to 300 microns and 400 microns). Other ranges are also possible.

In some embodiments, a microwell has a depth on the micron scale. In certain embodiments, this small depth results in smaller distances for analytes to travel to reach probes in the microwell, resulting in faster assays, as further described herein. In certain embodiments, this small depth also facilitates narrow probe articles (e.g., posts) to be fabricated within the microwell, with a probe article height less than or equal to the depth of the microwell, and to maintain structural stability due to a small height of the probe article. In some embodiments, a microwell has a depth of at least 1 micron, at least 5 microns, at least 10 microns, at least 20 microns, at least 25 microns, at least 30 microns, or at least 35 microns. In some embodiments, a microwell has a depth of at most 1000 microns, at most 500 microns, at most 100 microns, at most 50 microns, at most 40 microns at most 38 microns, or at most 36 microns. Combinations of the above-referenced ranges are also possible (e.g., between or equal to 1 micron and 1000 microns, between or equal to 10 microns and 40 microns, between or equal to 30 microns and 40 microns, between or equal to 30 microns and 38 microns). Other ranges are also possible.

A microwell is generally configured to contain a small volume. In certain embodiments, this small volume results in smaller distances for analytes to travel to reach probes in the microwell, resulting in faster assays, as further described herein. In some embodiments, a microwell is configured to contain a volume of at most 99 microliters, at most 50 microliters, at most 10 microliters, at most 1 microliter, at most 100 nL, at most 10 nL, at most 8 nL, at most 7 nL, at most 5 nL, or at most 4 nL. In some embodiments, a microwell is configured to contain a volume of at least 0.1 nL, at least 0.5 nL, at least 1 nL, at least 2 nL, or at least 3 nL. Combinations of the above-referenced ranges are also possible (e.g., between or equal to 0.1 nL and 99 nL, between or equal to 0.1 nL 50 microliters, between or equal to 0.1 nL and 10 nL, between or equal to 1 nL and 5 nL, between or equal to 3 nL and 4 nL). Other ranges are also possible. In some embodiments, a microwell is configured to contain a volume of less than 100 microliters and greater than or equal to 0.1 nL. It should be understood that other volumes (e.g., less than 0.1 nL, greater than or equal to 100 microliters) are also possible.

A microwell may have any suitable shape. In some embodiments, an interior surface of a microwell is cylindrical and/or has a circular cross-section. However, it should be understood that other shapes of the interior surface of a microwell (e.g., rectangular prism, pyramid) and/or cross-section (e.g., square, oval, rectangle, triangle) are also possible.

A microwell may comprise any suitable material. In some embodiments, a microwell (e.g., the walls and/or base of a microwell) comprises a polymer. Non-limiting examples of a polymer include polystyrene, polypropylene, polycarbonate, or a cyclo-olefin, or a combination thereof. In some embodiments, a microwell (e.g., the walls and/or base of a microwell) comprises glass and/or quartz. In some embodiments, a microwell comprises polymeric walls and a glass and/or quartz base. It should be understood that other materials are also possible.

Some apparatuses and methods described herein include one or more probe articles. As used herein, the term “probe article” refers to an article comprising a probe configured to detect an analyte (e.g., by capturing the analyte, by binding to the analyte). A probe article may be of any suitable size and shape for a given application.

In some embodiments, the probe article is a post. As used herein, the term “post” refers to a protrusion from a surface (e.g., from the base of a microwell). In other embodiments, the probe article is a free-standing article (e.g., a particle). A probe article (e.g., post) may be of any suitable size and shape for a given application. In some embodiments, the probe article (e.g., post) is cylindrical in shape (e.g., having a circular cross-section), but it should be understood that other shapes of the probe article (e.g., rectangular prism, pyramid) and/or cross-section (e.g., square, oval, rectangle, triangle) are also possible.

The probe article (e.g., post) may have a largest dimension (e.g., largest longitudinal dimension, largest transverse dimension, largest lateral dimension (e.g., diameter), height) on the micron scale. In some embodiments, the small size of the probe article increases the sensitivity of an assay that involves capturing an analyte with the probe article, as described further herein. In some embodiments, the probe article has a largest dimension of at least 1 micron, at least 5 microns, at least 10 microns, at least 20 microns, at least 25 microns, at least 30 microns, or at least 35 microns. In some embodiments, a probe article has a largest dimension of at most 1000 microns, at most 500 microns, at most 400 microns, at most 300 microns, at most 200 microns, at most 100 microns, at most 80 microns, at most 60 microns, or at most 40 microns. Combinations of the above-referenced ranges are also possible (e.g., between or equal to 1 micron and 1000 microns, between or equal to 1 microns and 100 microns, between or equal to 1 microns and 40 microns, between or equal to 30 microns and 40 microns). Other ranges are also possible.

In some embodiments, a probe article (e.g., post) has any suitable aspect ratio. As used herein, “aspect ratio” refers to the ratio of the largest longitudinal dimension (e.g., height) to the largest transverse dimension (e.g., diameter) of a probe article. In some embodiments, a probe article has an aspect ratio of at least 1, at least 1.5, or at least 2. In some embodiments, a probe article has an aspect ratio of at most 1000, at most 500, at most 100, at most 50, at most 40, at most 30, at most 20, at most 10, or at most 5. Combinations of the above-referenced ranges are also possible (e.g., between or equal to 1 and 1000, between or equal to 1 and 40, between or equal to 1 and 5). Other ranges are also possible.

The probe article (e.g., post) may have a largest transverse dimension (e.g., diameter) on the micron scale. In some embodiments, the small largest transverse dimension of the probe article increases the sensitivity of an assay that involves capturing an analyte with the probe article. For example, in embodiments where a probe article is a post protruding from a substrate (e.g., from the base of a microwell), the largest transverse dimension of the probe article is referred to herein as the largest lateral dimension of the probe and is measured along a plane parallel to the substrate. For example, in embodiments where a probe article is a post protruding from a substrate (e.g., from the base of a microwell), and an assay involves measuring a quantity (e.g., a fluorescence intensity) indicative of a quantity of captured analyte, the largest transverse dimension of the probe article is measured along a plane parallel to the substrate and perpendicular to the direction of measurement from the measuring device (e.g., photodetector) to the probe article. In some embodiments, the probe article has a largest transverse dimension of at least 1 micron, at least 5 microns, at least 10 microns, at least 20 microns, at least 25 microns, at least 30 microns, or at least 35 microns. In some embodiments, a probe article has a largest transverse dimension of at most 1000 microns, at most 500 microns, at most 400 microns, at most 300 microns, at most 200 microns, at most 100 microns, at most 80 microns, at most 60 microns, or at most 40 microns. Combinations of the above-referenced ranges are also possible (e.g., between or equal to 1 micron and 1000 microns, between or equal to 1 microns and 100 microns, between or equal to 1 microns and 40 microns, between or equal to 30 microns and 40 microns). Other ranges are also possible.

The probe article (e.g., post) may have a largest transverse cross-sectional area (e.g., largest lateral cross-sectional area) on the micron scale. In some embodiments, the small largest transverse cross-sectional area of the probe article increases the sensitivity of an assay that involves capturing an analyte with the probe article. For example, in embodiments where a probe article is a post protruding from a substrate (e.g., from the base of a microwell), the largest transverse cross-sectional area of the probe article is referred to herein as the largest lateral cross-sectional area of the probe and is measured along a plane parallel to the substrate. In some embodiments, the probe article has a largest transverse cross-sectional area of at least 0.1 micron², at least 1 micron², at least 10 micron², or at least 100 micron². In some embodiments, the probe article has a largest transverse cross-sectional area of at most 6000 micron², at most 4000 micron², at most 2000 micron², at most 1300 micron², at most 1000 micron², at most 800 micron², at most 600 micron², at most 400 micron², at most 300 micron², or at most 200 micron². Combinations of the above-referenced ranges are also possible (e.g., between or equal to 0.1 micron² and 6000 micron², between or equal to 0.1 micron² and 1300 micron², between or equal to 0.1 micron² and 300 micron²). Other ranges are also possible.

In some embodiments, a probe article is a post in a microwell described herein. In some such embodiments, a probe-containing post has a height less than or equal to the height of the corresponding microwell in which it resides (e.g., 35 microns, 36 microns, 38 microns).

In certain embodiments, a method of fabricating a probe-containing post in a microwell involves delivering a prepolymer solution into the microwell, placing a material (e.g., a PDMS slab) onto the microwell such that the prepolymer solution fills the microwell at its depth but not above its depth, and exposing the prepolymer solution to electromagnetic radiation (e.g., through a photomask) in a portion of the microwell such that a post is formed protruding from the base of the microwell and equal in height to the depth of the microwell.

In some embodiments, a probe-containing post protrudes from a base of a microwell. In some embodiments, a probe-containing post protrudes from a wall of a microwell.

In certain embodiments, probe articles with dimensions as described herein have a low total sensing surface and a high surface area to volume ratio, which facilitate analysis of biological samples comprising an analyte at a far lower mass (e.g., on the order of 100 times lower) than samples that were able to be analyzed using alternative apparatuses and methods. In some embodiments where the analyte is a nucleic acid (e.g., miRNA), these probe articles facilitate analysis of the analyte at low mass without the need for signal amplification.

A probe article (e.g., post) may have any suitable shape. In some embodiments, a probe article is cylindrical and/or has a circular cross-section. However, it should be understood that other shapes of the probe article (e.g., rectangular prism, pyramid) and/or cross-section (e.g., square, oval, rectangle, triangle) are also possible.

In some embodiments, a probe article (e.g., post) comprises a polymer. In some embodiments, a probe article (e.g., post) comprises a hydrogel. As used herein, the term “hydrogel” refers to an article comprising a crosslinked polymer mesh and an aqueous medium (comprising water) within the crosslinked polymer mesh. In certain embodiments, a probe article is a hydrogel post protruding from a substrate (e.g., from the base of a microwell. In some embodiments, the polymer of the probe article and/or crosslinked polymer mesh of the hydrogel probe article comprises polyethylene glycol (PEG). A probe article generally comprises a material which is non-fouling, that does not adhere to cells or other biological materials, and a probe configured to detect an analyte (e.g., to capture an analyte, that specifically binds to an analyte).

In some embodiments, a method of forming a probe article comprises exposing at least a portion of an aqueous solution comprising polyethylene glycol diacrylate (PEGDA) and a photoinitiator (e.g., (2-hydroxy-2-methylpropiophenone)), one or more probes (e.g., a nucleic acid probe, a DNA probe), and/or a probe solubilizer and/or stabilizer (e.g., TE buffer for a nucleic acid probe, e.g., for a DNA, cDNA, or RNA probe) to electromagnetic irradiation such that a probe article comprising polyethylene glycol and one or more probes is formed. In some embodiments, a probe is contained within the mesh of the hydrogel of the probe article, e.g., by the probe having a size larger than the mesh size of the hydrogel. In some embodiments, a probe is bound to the probe article (e.g., post) by any suitable intermolecular interaction, e.g., covalent bonding, metallic bonding, dipole-dipole interactions, hydrophobic interactions, Van der Waals interactions, pi-pi stacking, or any other suitable intermolecular interaction.

A probe article (e.g., post) generally comprises a probe configured to detect an analyte. As used herein, the term “analyte” will be understood by those of ordinary skill in the art and refers to a molecule (e.g., a small molecule, an oligomer, a polymer, a nucleic acid, an miRNA) or a cell (e.g., a Calu-6 cell) that is the subject of an analysis (e.g., an assay, a chemical analysis, a biochemical analysis, a biological analysis). In some embodiments, an analyte comprises a nucleic acid (e.g., a microRNA (miRNA)). In some embodiments, the analyte comprises a miRNA. In some embodiments, the miRNA of the analyte comprises let-7, miR-34, miR-21, or miR-155. However, it should be understood that any suitable analyte (e.g., nucleic acid, protein, cytokine, cell) is also possible.

In some embodiments, an analyte comprises a cell (e.g., a Calu-6 cell) or molecule from a biological sample. As used herein, the term “biological sample” is a sample (e.g., cell sample, tissue sample, tissue section, serum sample) from a living organism, which may or may not comprise a cell or a plurality of cells. In some embodiments, the biological sample comprises a diseased cell and/or is from a living organism having a disease state. A non-limiting example of a disease state is a cancer. A non-limiting example of a cancer is human non-small-cell lung carcinoma (NSCLC). In some embodiments, a diseased cell is a cancerous cell. A non-limiting example of a cancerous cell is a human non-small-cell lung carcinoma (NSCLC) cell.

In some embodiments, a probe is configured to detect an analyte by capturing the analyte. In some embodiments, the probe is configured to capture the analyte by binding to the analyte. Binding of the analyte to the probe may be by any suitable intermolecular interaction, e.g., covalent bonding, metallic bonding, dipole-dipole interactions, hydrophobic interactions, Van der Waals interactions, pi-pi stacking, or any other suitable intermolecular interaction.

In some embodiments, a probe is configured to hybridize with a nucleic acid of the analyte. In some embodiments, a probe comprises a nucleic acid. In some embodiments, a probe comprises a deoxyribonucleic acid (DNA) probe. In some embodiments, a probe comprises a nucleic acid having at least a portion complimentary to a nucleic acid of an analyte. In some embodiments, a probe has at least a portion configured to hybridize with a microRNA (miRNA). In some embodiments, a probe comprises a deoxyribonucleic acid (DNA) probe having a portion complimentary to an miRNA analyte.

In some embodiments, a probe comprises a ligand configured for binding a cell surface receptor.

In some embodiments, a probe article (e.g., post) comprises a plurality of a single probe type, also referred to herein as a plurality of a first probe, wherein each first probe is configured to detect a first analyte. In some embodiments, a plurality of probe types (e.g., two, three, four, or more probe types) are present on a single probe article, wherein each probe type is configured to detect a different corresponding analyte type. For example, a probe article may have probe A and probe B, configured to detect analyte A′ and analyte B′ respectively, where probe A differs from probe B and analyte A′ differs from analyte B′. For example, a probe article might include probe A and probe B, wherein probe A and probe B are each configured to capture (e.g., bind) a corresponding analyte type and a corresponding adapter (e.g., biotinylated adapter) that could be used to attach a corresponding labelling article (e.g., fluorophore) to allow spectral multiplexing within the probe article. This might be particularly advantageous, e.g., if a microwell contains a maximum number of probe articles (e.g., posts) per well that physically fit into the microwell and even higher multiplexing is needed. As used herein, to be of a different “type” or to “differ” may mean chemically different (e.g., a different miRNA) in the case of analyte molecules, or different in cell type in the case of analytes that comprise cells.

In some embodiments, a microwell comprises a plurality of probe articles (e.g., posts) in the microwell. In certain embodiments, each probe article comprises a corresponding probe type configured to detect a corresponding analyte type. For example, a probe article A may have probe A1 configured to detect analyte A1′, and a probe article B may have probe B1 configured to detect analyte B1′, where probe A1 differs from probe B1 and analyte A1′ differs from analyte B1′.

In some embodiments, a microwell comprises any suitable number of probe articles (e.g., posts). In some embodiments, a microwell comprises between or equal to 1 and 100 probe articles (e.g., posts) (e.g., between or equal to 2 and 50, between or equal to 3 and 10; e.g., 1, 2, 3, 4, 5, 6, 50). For example, in certain embodiments, for posts 20 microns in diameter and 20 microns spaced between each post, about 50 posts may be present within a single microwell that is 300 microns in diameter, wherein each post is protruding from the base of the microwell.

In some embodiments, a microwell comprises a plurality of posts. In some embodiments, each post has a corresponding probe type configured to detect a corresponding analyte type. For example, in some embodiments, a microwell comprises a plurality of posts, each post having a probe complementary to a different corresponding nucleic acid (e.g., miRNA) analyte. In some embodiments, each post is physically separate from each other post in the microwell. In some embodiments, a post is spaced from another post by at least 0.1 micron and at most 998 microns (e.g., at least 1 micron and at most 200 microns, at least 1 micron and at most 50 microns).

In some embodiments, a microwell contains one probe article (e.g., post) per probe type. In some embodiments, a microwell contains a plurality of probe articles (e.g., post) for a given probe type. In certain embodiments, it may be advantageous to have a single probe article per probe type in a microwell in order to increase the sensitivity of an assay for an analyte detected by the probe type.

In some embodiments, an apparatus comprises a microwell array. As used herein, the term “array” refers to an ordered arrangement of a plurality of articles in one dimension (e.g., in a linear arrangement), two dimensions (e.g., in a planar arrangement), or three dimensions (e.g., a plurality of layers of microwell sub-arrays).

A microwell array comprises any suitable number of microwells. In some embodiments, apparatuses and methods described herein allow for high throughput analysis of samples in microwell arrays described herein, analyzing the contents of a large number of microwells simultaneously. In some embodiments, a microwell array comprises at least 1, at least 2, at least 3, at least 4, at least 5, at least 10, at least 100 microwells. In some embodiments, a microwell array comprises at most 1,000,000, at most 100,000, at most 10,000, or at most 1000 microwells. Combinations of the above-referenced ranges are also possible (e.g., between or equal to 1 and 1,000,000 microwells, between or equal to 1 and 1000 microwells, between or equal to 100 and 10,000 microwells). Other ranges are also possible.

A microwell array may have any suitable size. In some embodiments, a microwell array has an area of between or equal to 25 microns squared and 1 m² (e.g., between or equal to 100 microns squared and 100 cm², between or equal to 1 mm² and 10 cm²; e.g., 1 cm²). Other ranges are also possible. In embodiments where the microwell array is a plurality of layers of microwell sub-arrays of the same area, the area of the microwell array is equal to the area of a microwell sub-array multiplied by the number of layers.

In some embodiments, a microwell array is configured such that the contents of each microwell in the microwell array are physically isolated from the contents of at least one (e.g., every) other microwell in the microwell array.

In some embodiments, an apparatus comprises a microwell and/or microwell array. In some embodiments, the apparatus further comprises a substrate. In some embodiments, a substrate is arranged, relative to a microwell array, as an enclosing structure for the microwell array, such that the contents of each microwell in the microwell array are physically separated from the contents of every other microwell in the microwell array. In some embodiments, an apparatus comprises a microwell and/or microwell array and an enclosing structure (e.g., substrate, microwell array, tissue sample) that at least partially (e.g., completely) physically separates each microwell from an external environment to the microwell, to form an enclosure (e.g., a reactor) in each microwell.

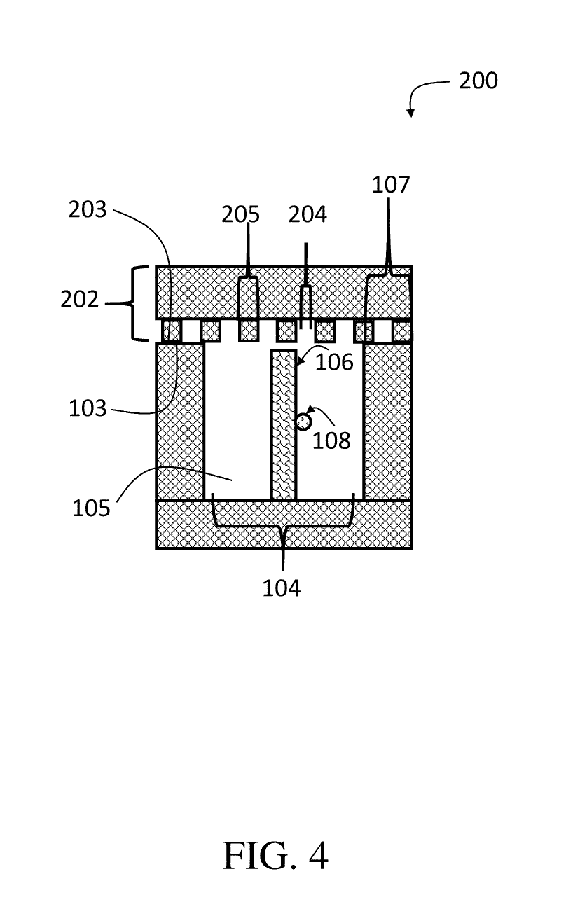

In some embodiments, a surface of the substrate is proximate a surface of the microwell and/or the microwell array (e.g., FIG. 4). As used herein, the term “proximate” refers to the positioning of a first surface (e.g., of a substrate) close enough to a second surface (e.g., of a microwell and/or microwell array) so as to form an enclosure (e.g., comprising the interior surface of a first microwell array and the interior surface of a second microwell array, comprising the interior surface of a first microwell array and a surface of a tissue sample), e.g., allowing the contents of the first surface to interact with the contents of the second surface. Some methods described further herein comprise positioning a surface of a substrate, wetted with a liquid containing an agent, proximate a surface of a microwell array, such that the liquid containing the agent can mix with any fluid present in at least some of the microwells, and such that the contents of each microwell are physically isolated from the contents of every other microwell in the array. For example, in embodiments wherein the substrate comprises microwells and a liquid comprising an agent wets the substrate and is held in the microwells by capillary action and surface tension, proximate may be on the order of 0.1 microns or less (e.g., contacting). In certain embodiments, a surface of a substrate (e.g., a surface of a microwell array) is positioned in contact with a surface of a first microwell array in order to accomplish physically separating the contents of each microwell in the first microwell array from one another. As another example, in embodiments wherein the substrate comprises a tissue sample (e.g., a tissue section) fixed to the substrate, proximate may be on the order of the tissue sample thickness (e.g., between or equal to 5 microns and 100 microns).

In some embodiments, an apparatus comprises a first microwell array and a second microwell array sandwiched together, with a surface of the first microwell array proximate (e.g., contacting) a surface of the second microwell array such that the contents of each microwell in the first microwell array are separated from the contents of each other microwell in the first microwell array.

In some embodiments, a largest lateral dimension (e.g., a diameter) of the microwells in the second microwell array is less than a largest lateral dimension (e.g., a diameter) of the microwells in the first microwell array. For example, in some embodiments, a largest lateral dimension (e.g., a diameter) of the microwells in the second microwell array is between or equal to 0.01 and 0.2 times (e.g., 0.1 times) a largest lateral dimension (e.g., a diameter) of the microwells in the first microwell array. Other multiples are also possible. For example, an apparatus comprises a first microwell array having microwells with a largest lateral dimension of 300 microns, sandwiched together with a second microwell array having microwells with a largest lateral dimension of 30 microns.

In some embodiments, a spacing between the microwells in the second microwell array is less than a spacing between the microwells in the first microwell array. For example, in some embodiments, a spacing between the microwells in the second microwell array is between or equal to 0.01 and 0.2 times (e.g., 0.1 times) a spacing between the microwells in the first microwell array. Other multiples are also possible. For example, an apparatus comprises a first microwell array having a spacing between the microwells of 300 microns, sandwiched together with a second microwell array having a spacing between the microwells of 30 microns.

In some embodiments, a spacing between microwells in a microwell array is between or equal to 0.5 and 5 times (e.g., equal to) a largest lateral dimension of microwells in a microwell array. Other multiples are also possible.

In some embodiments, an apparatus comprises a microwell array and a substrate comprising a tissue sample (e.g., a tissue section), wherein a surface of the tissue sample is proximate a surface of the microwell array.

In some embodiments, an apparatus comprises a substrate and a microwell and/or microwell array that come together with proximate surfaces to form a reactor or a plurality of reactors, each reactor comprising each microwell. In some embodiments, the reactor has a volume of between or equal to 0.1 nanoliter and 100 microliters (e.g., between or equal to 0.1 nanoliter and 100 nanoliters, between or equal to 1 nanoliter and 10 nanoliters; e.g., 3.2 nL). Other ranges are also possible.

In some embodiments, an apparatus comprises a biological sample, e.g., proximate a surface of a microwell or microwell array. In some embodiments, an apparatus comprises an aqueous medium comprising one or more agents (e.g., a cell lysis agent, an extraction agent for an analyte, a capture agent for an analyte), e.g., at least partially contained within a microwell or microwell array. In some embodiments, a probe article (e.g., post) in a microwell comprises a probe, a captured analyte (e.g., bound analyte), an adapter, a ligase, and/or a labelling article (e.g., fluorophore) in order to detect the captured analyte (e.g., by fluorescence microscopy).

A probe article (e.g., post) in a microwell may be formed by any suitable method. Methods of fabricating one or more posts in a microwell are provided. In some embodiments, a method of fabricating a post in a microwell comprises exposing an interior of the microwell to a first prepolymer solution (e.g., comprising a prepolymer, a probe, a photoinitiator, and/or a probe solubilizer/stabilizer). In some embodiments, the method further comprises exposing a first portion of the interior of the microwell to electromagnetic radiation to form a first post. For example, a method of fabricating a post in a microwell may involve exposing an interior of the microwell to a prepolymer solution and then exposing a first portion of the interior of the microwell to electromagnetic radiation (e.g., through a photomask) to form a first post. In some embodiments, the method further comprises washing the interior of the microwell. In some embodiments, the method further comprises exposing the interior of the microwell to a second prepolymer solution. In some embodiments, the method further comprises exposing a second portion of the interior of the microwell to electromagnetic radiation to form a second post. Any component (e.g., a prepolymer, a probe, a photoinitiator, and/or a probe solubilizer/stabilizer) of the first prepolymer may be chemically the same or different from any corresponding component of the second prepolymer.

A microwell array may be formed by any suitable method. For example, a microwell array may be formed by filling a mold (e.g., a polydimethylsiloxane (PDMS) mold) with a photocuring polymer (e.g., adhesive). The method may further comprise contacting the photocuring polymer with a substrate (e.g., glass, acrylated glass). The method may further comprise exposing the photocuring polymer to electromagnetic radiation such that the photocuring polymer cures to form a solid structure attached to the substrate.

Methods of assaying an analyte in a biological sample are provided. In some embodiments, a method involves exposing a biological sample (e.g., as described herein) to a probe (e.g., as described herein) on a post (e.g., as described herein) in a microwell (e.g., as described herein), wherein the probe is configured to detect an analyte (e.g., as described herein).

In some embodiments, a method comprises bringing a biological sample proximate a surface of a microwell or microwell array.

In some embodiments, bringing a biological sample proximate a surface of a microwell or microwell array comprises settling cells (e.g., Calu-6 cells) into the bottom of a microwell comprising a post or a plurality of posts, or into the bottom of microwells (e.g., comprising one or more posts) in a microwell array. In certain embodiments, an average of between or equal to 10 and 300 cells per well are settled in a microwell array (e.g., 14, 50, 110, 200 cells per microwell array). In some embodiments, cells are settled for a short period of time (e.g., at most 30 minutes, at most 10 minutes).

Exposing a biological sample to a probe on a post in a microwell may be different depending on the type of probe. For example, in some embodiments, (e.g., where a probe comprises a cell capture agent (e.g., a ligand configured to bind a cell surface receptor)), exposing a biological sample to a probe on a post in a microwell comprises settling cells into the microwell. In some embodiments where a probe is configured to detect a biological molecule (e.g., nucleic acid) obtained by cell lysis, exposing a biological sample to a probe on a post in a microwell comprises bringing a liquid comprising a cell lysis agent proximate a cell sample or tissue sample. In some embodiments, this can be accomplished by settling cells in the microwell and then bringing a liquid comprising a cell lysis agent proximate the microwell (e.g., by wetting a substrate with the liquid and bringing the substrate proximate the microwell), such that the cell lysis agent can interact with the settled cells. In some embodiments, this can be accomplished by at least partially filling a microwell with a liquid comprising a cell lysis agent and bringing a surface of a tissue sample proximate a surface of the microwell, such that the cell lysis agent can interact with the tissue sample.

In embodiments herein, any apparatus or method involving a microwell can equally involve a microwell array.

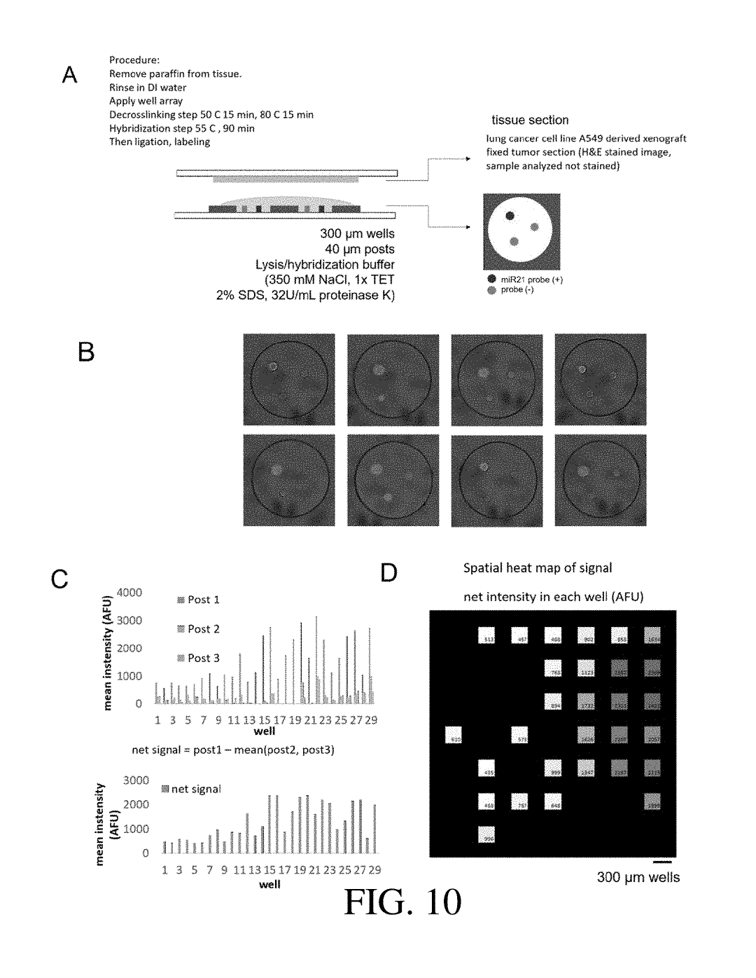

In some embodiments, bringing a biological sample proximate a surface of a microwell or microwell array comprises bringing a surface of a tissue sample (e.g., tissue section) proximate a surface of the microwell or microwell array. In some embodiments, the tissue sample is a formalin-fixed, paraffin-embedded (FFPE) tissue sample. In some embodiments, a method comprises removing the paraffin from a fixed tissue sample (e.g., using xylenes or other organic solvent(s)) and rinsing the tissue in deionized water, before bringing the surface of the tissue sample proximate the surface of the microwell or microwell array. In some embodiments, a method involves bringing a surface of the tissue sample proximate the surface of the microwell or microwell array without first removing the paraffin. In some embodiments, a method further involves a de-crosslinking step to reverse the formalin crosslinks in an FFPE tissue sample, for a suitable set of temperatures to which the tissue sample is exposed (e.g., between or equal to 30 degrees Celsius and 60 degrees Celsius (e.g., 50 degrees Celsius); followed by e.g., between or equal to 70 degrees Celsius and 90 degrees Celsius (e.g., 80 degrees Celsius) and durations (e.g., between or equal to 1 minute and 30 minutes (e.g., 15 minutes); followed by e.g., between or equal to 1 minute and 30 minutes (e.g., 15 minutes)) (e.g., FIG. 10A). Other ranges are also possible.

In some embodiments, a method comprises delivering one or more agents (e.g., two, three, or more agents) to a microwell and/or microwell array. Non-limiting examples of agents include a cell lysis agent (e.g., sodium dodecyl sulfate (SDS), e.g., in a lysis buffer), an extraction agent for an analyte (e.g., an miRNA extraction agent, e.g., proteinase K) and/or a capture agent for an analyte (e.g., a hybridization agent (e.g., in a hybridization buffer)). For example, in some embodiments, a method comprises delivering a hybridization agent to a microwell and/or microwell array to hybridize a nucleic acid analyte with a probe on a probe article (e.g., post) in one or more microwells (e.g., each microwell). In some embodiments, a cell lysis agent (also referred to herein as a lysis agent) comprises sodium dodecyl sulfate (SDS). In some embodiments, an extraction agent comprises proteinase K. In some embodiments, a capture agent for an analyte comprises a hybridization agent.

In some embodiments, one or more agents are delivered by a liquid comprising the one or more agents. In some embodiments, the liquid comprises a hybridization buffer. In certain embodiments, SDS is included in a liquid (e.g., buffer) not only to lyse cells, but along with proteinase K also to free miRNA from its associated protein complexes.

As used herein, the term “lysis buffer” will be known to those of ordinary skill in the art and refers to an aqueous solution configured to lyse cells (e.g., animal cells, human cells, plant cells), the solution comprising a weak acid and its conjugate base, and a lysis agent and/or a salt and/or a detergent.

As used herein, the term “lysis agent” will be known to those of ordinary skill in the art and refers to an agent configured to lyse cells.

As used herein, the term “hybridization buffer” will be known to those of ordinary skill in the art and refers to an aqueous solution configured to hybridize complementary nucleic acids.

In some embodiments (e.g., in a cell assay), delivery of the one or more agents comprises wetting a substrate (e.g., comprising microwells) with a liquid comprising the one or more agents and bringing a surface of the substrate proximate to a surface of the microwell and/or microwell array (e.g., in which the cells settled).

In some embodiments (e.g., in a tissue assay), delivery of the one or more agents comprises at least partially (e.g., completely) filling the one or more microwells (e.g., each comprising one or more probe-containing posts) in a microwell array with a liquid comprising the one or more agents. In some embodiments, a method further comprises positioning a surface of a tissue sample (e.g., a tissue section) proximate a surface of the microwell array such that the one or more agents in the fluid can interact with the tissue sample.

In some embodiments, a method involves incubating a microwell (e.g., comprising one or more posts) or microwell array with a biological sample (e.g., cell sample, tissue sample). Other materials incubated with the microwell or microwell array and biological sample may include but are not limited to a liquid comprising one or more agents (e.g., a cell lysis agent) and a substrate having a surface positioned proximate a surface of the microwell or microwell array. Incubation may in some embodiments be carried out for cell lysis, analyte (e.g., miRNA) extraction, and analyte capture (e.g., hybridization). Incubation may occur for any suitable duration (e.g., between or equal to 30 minutes and 120 minutes, e.g., 90 minutes) at any suitable temperature range (e.g., between or equal to 30 degrees Celsius and 80 degrees Celsius, 55 degrees Celsius). Other ranges are also possible.

In some embodiments, a method involves lysing a cell by exposing the cell to a liquid comprising a lysis agent (e.g., a lysis buffer). Exposing the cell to a liquid comprising a lysis agent may comprise wetting a first substrate with the liquid and positioning a surface of the first substrate proximate to a surface of the microwell such that the contents of the microwell are physically isolated from an exterior of the microwell.

In some embodiments, a method comprises capturing an analyte with a probe on a post in a microwell. In some embodiments, capturing an analyte involves binding a cell surface receptor on the cell analyte to a ligand probe. In some embodiments, capturing an analyte involves capturing the analyte from a lysed cell. In some embodiments, capturing an analyte involves hybridizing the analyte (e.g., a nucleic acid analyte) with a probe (e.g., a nucleic acid probe complementary to a nucleic acid analyte).

In some embodiments, a method comprises a washing step, which may comprise washing a probe article (e.g., post) after capturing the analyte, e.g., to remove biological material, agents, and other materials not bound to the probe article.

In some embodiments, a method comprises a ligation step, which comprises exposing a probe article (e.g., post) to a ligation solution after capturing the analyte (e.g., after washing the probe article). A ligation solution generally comprises an adapter (e.g., configured to bind to a fluorophore) and a ligase configured to ligate the adapter with a portion of a probe, which probe has captured an analyte. A ligase may be configured not to ligate the adapter with a portion of a probe when the probe has not captured an analyte.

In some embodiments, a method comprises a washing step, which may comprise washing a probe article (e.g., post) after ligation, e.g., to remove any ligase, adapter, and other materials not bound to the probe article.

In some embodiments, a method comprises a labeling step, which comprises exposing a probe article (e.g., post) to a labeling solution after ligation (e.g., after washing the probe article). A labeling solution may comprise a labelling article. A labeling solution may comprise a labelling article, e.g., a fluorophore which binds to the adapter. A labeling solution may comprise a labelling article, e.g., a digoxigenin or a radioactive probe. The method may further comprise fluorescently labeling probes that captured analyte with a fluorophore, e.g., by binding the fluorophore with the adapter. In certain embodiments, the measured fluorescence signal from the probe article(s) (e.g., posts) (e.g., in the microwells) is directly proportional to the amount of analyte present in the portion of the biological sample proximate the probe article(s).

In some embodiments, a method comprises measuring a fluorescence intensity of a probe article (e.g., post) (e.g., after a labeling step). In some embodiments, the fluorescence intensity measured is an average fluorescence intensity. For example, in embodiments where a probe article is a post protruding from a substrate (e.g., from the base of a microwell) and fluorescence intensity is measured in a plane parallel to the substrate, the fluorescence intensity measured is an average fluorescence intensity across the cross-sectional area of the post.

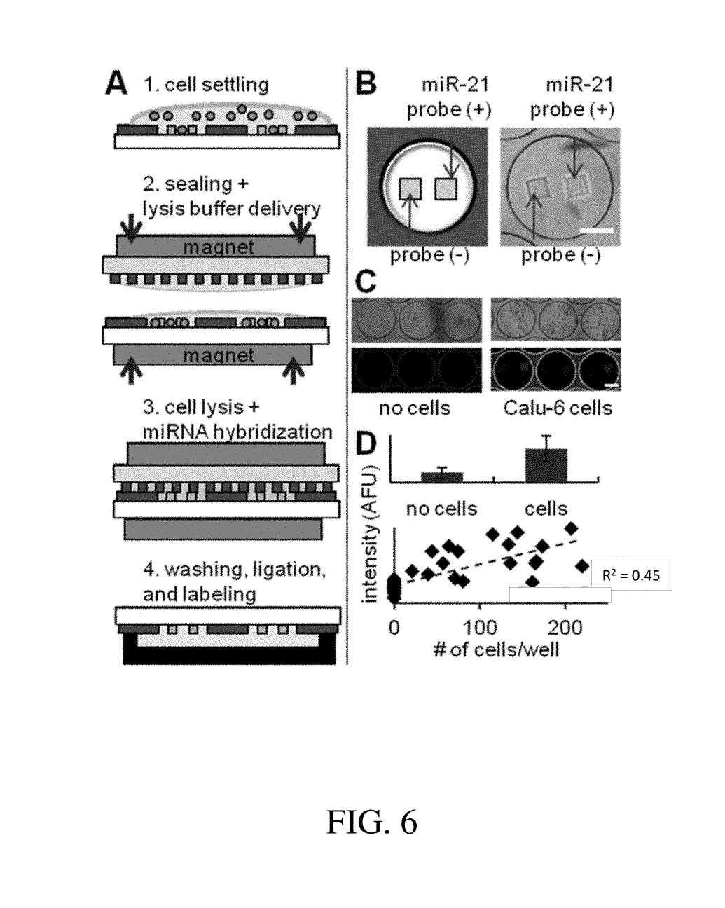

In some embodiments, a method involves maintaining the positioning of a surface of a substrate proximate a surface of a microwell or microwell array, and/or maintaining the relative lateral position of the substrate and the microwell or microwell array, during an assay so as to retain spatial information (e.g., differentiating a first portion of a biological sample at the position of one microwell in a microwell array from another portion of the biological sample at the position of another microwell in the microwell array). Maintaining the positioning of the surface of the substrate proximate the surface of the microwell or microwell array, and/or maintaining the relative lateral position of the substrate and the microwell or microwell array, may comprise clamping the substrate to the microwell array using a clamp. Maintaining the positioning of the surface of the substrate proximate the surface of the microwell or microwell array, and/or maintaining the relative lateral position of the substrate and the microwell or microwell array, may comprise positioning magnets on the outside of the substrate-microarray sandwich, at the base of the microwell array on the opposite side of the array relative to the microwell(s) and on the opposite side of the substrate to that facing the microwell or microwell array (e.g., FIG. 6A, 2.).

In some embodiments, an assay described herein has a lower limit of detection of at most 800 cells per well (e.g., at most 100 cells per well, at most 20 cells per well) and at least 10 cells per well (e.g., 16 cells per well).

In some embodiments, a method of assaying an analyte in a tissue sample (e.g., tissue section) is provided. In some embodiments, the method involves positioning separate probe articles proximate to (e.g., within 40 microns of) separate areas of a surface of the tissue sample. In some embodiments, the method involves contacting separate probe articles to separate areas of a surface of the tissue sample. Separate probe articles may have a spacing as described herein. In some embodiments, a method comprises positioning, essentially simultaneously, separate probe articles proximate to separate areas of a surface of the tissue sample. In some embodiments, at least some of the separate probe articles are configured to detect a first analyte. Any suitable number of probe articles may be used for detecting any suitable number of analytes from the tissue sample. In some embodiments, at least some of the separate probe articles are posts attached to a common substrate (e.g., the base of one or more microwells in a microwell array).

In some embodiments, a method comprises delivering one or more agents (e.g., two, three, or more agents; e.g., an agent as described herein) to the surface of the tissue sample and/or at least some of the separate probe articles. For example, in some embodiments, a method comprises delivering a hybridization agent to a tissue sample and/or at least some of the separate probe articles to hybridize a nucleic acid analyte with a probe on at least some of the separate probe articles (e.g., posts).

In some embodiments, a method comprises capturing an analyte from a tissue sample (e.g., tissue section) with at least some of the separate probe articles. In some embodiments, the analyte is captured by a probe article located nearer (e.g., nearest), relative to the other separate probe articles, a portion of the tissue section from which the analyte originated.

In some embodiments, a method comprises lysing at least some cells in a tissue sample (e.g., tissue section) by exposing the surface of the tissue section to a liquid comprising a lysis agent while maintaining the separate probe articles proximate to the separate areas of the surface of the tissue section.

In some embodiments, an analyte is a nucleic acid, and capturing the analyte comprises hybridizing the nucleic acid with a probe on at least some of the separate probe articles. In some embodiments, the analyte is a microRNA.

In certain embodiments, an assay has a low lower limit of detection (LLOD) without signal amplification. In certain embodiments, an assay is able to detect a significantly lower amount of analyte as compared with other assay methods and apparatus. In certain embodiments, an assay has a lower limit of detection (LLOD) of 0.004 amol (e.g., at least 0.025 amol). In certain embodiments, an assay has a lower limit of detection of between or equal to 0.004 amol and 1 amol (e.g., between or equal to 0.025 amol and 0.1 amol, between or equal to 0.025 amol and 1 amol). Other ranges are also possible.

In certain embodiments, an assay has a large dynamic range. As used herein, the term “dynamic range” of an assay refers to the ratio between the largest and smallest values (e.g., of arbitrary fluorescence units) that can be detected during the assay. It may be important to have a wide dynamic range if the assay is a multiplex assay having more than one target analyte, since expression levels can vary across orders of magnitude for different target analytes (e.g., different miRNA targets). This may be particularly helpful in instances where a cell or tissue sample is limited in quantity. In some embodiments, the assay has a dynamic range of between or equal to 100 and 1,000,000 (e.g., between or equal to 1000 and 100,000, between or equal to 1000 and 10,000; e.g., 1000, 10,000). Other ranges are also possible.

In certain embodiments, assays on biological samples can be carried out on unprocessed cell samples and/or tissue samples with no prior sample preparation (e.g., no prior nucleic acid extraction).

In certain embodiments, assays on biological samples can quantitatively assay an analyte from samples with a small number of cells (e.g., below 1000 cells).

In some embodiments, methods of delivering an agent (e.g., a reagent; e.g., a plurality of agents) are provided.

In some embodiments, a method comprises wetting a substrate with a liquid comprising the agent. In some embodiments, wetting involves depositing a liquid containing an agent (e.g., an agent described herein) onto the substrate by any suitable liquid deposition method (e.g., dropping, flowing, spraying, spin-coating).

In some embodiments, a method comprises settling cells into a microwell or microwell array.

In some embodiments, a method comprises introducing probe articles (e.g., posts) to at least some microwells in a microwell array. Probe articles may be introduced by settling free-standing probe articles (e.g., particles) into a microwell array. Probe articles may be introduced by fabricating probe-containing posts in a microwell array (e.g., posts protruding from the base and/or walls of the microwell(s)).

In some embodiments, a method comprises positioning a surface of a substrate proximate to a surface of a first microwell array. In some embodiments, a method comprises maintaining (e.g., using a magnet, using a clamp) the positioning of the substrate relative to the first microwell array.

In some embodiments, a method comprises positioning a surface of a substrate proximate to a surface of a first microwell array such that the contents of each microwell in the first microwell array are physically separated from one another. In some embodiments, positioning the surface of a substrate proximate to a surface of a first microwell array creates sealed enclosures (e.g., reactors) having a small volume (e.g., between or equal to 0.1 nL and 50 microliters, between or equal to 0.1 nL and 10 nL). Other ranges are also possible.

In some embodiments, a substrate comprises a second microwell array. In some embodiments, the microwells of the second microwell array have a largest lateral dimension (e.g., diameter) smaller than the largest lateral dimension (e.g., diameter) of the microwells of the first microwell array. In some embodiments, the second microwell array has a spacing between the microwells smaller than the spacing between the microwells of the first microwell array. In some embodiments, the second microwell array has a spacing between the microwells smaller than the spacing between the microwells of the first microwell array, and the microwells of the second microwell array have a largest lateral dimension (e.g., diameter) smaller than or equal to the spacing between the microwells of the first microwell array.

Turning now to the figures, several non-limiting embodiments are described in further detail. However, it should be understood that the current disclosure is not limited to only those specific embodiments described herein. Instead, the various disclosed components, features, and methods may be arranged in any suitable combination as the disclosure is not so limited.

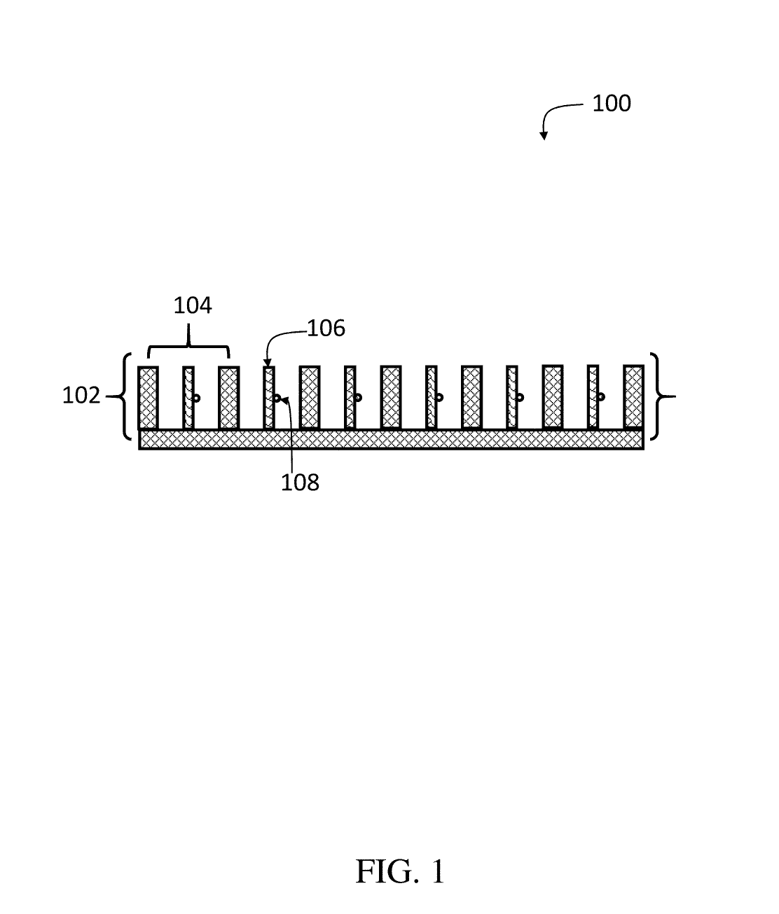

FIG. 1 is a side cross-sectional view schematic of an apparatus 100 for cell and/or tissue assays, according to some illustrative embodiments. The depicted apparatus 100 includes a microwell array 102. Each depicted microwell 104 in the microwell array 102 includes a post 106 in the microwell 104. The post 106 comprises a probe 108 configured to detect an analyte. It should be understood that this schematic is non-limiting and any suitable cross-section shape of the posts and of the microwells is possible. While FIG. 1 depicts an array of six (6) microwells, it should be understood that any suitable number of microwells (e.g., between or equal to 2 microwells and 100,000 microwells) in an array (e.g., a one-dimensional array, a two-dimensional array) are possible. The probe 108 may protrude from the post 106 (e.g., from a solid post) as depicted or may be embedded in the post 106 (e.g., in a gel post), as the disclosure is not so limited. The post 106 may comprise a plurality of probes 108, e.g., distributed throughout the surface and/or volume of the post 106. The microwells may each comprise a plurality of posts and/or probes.

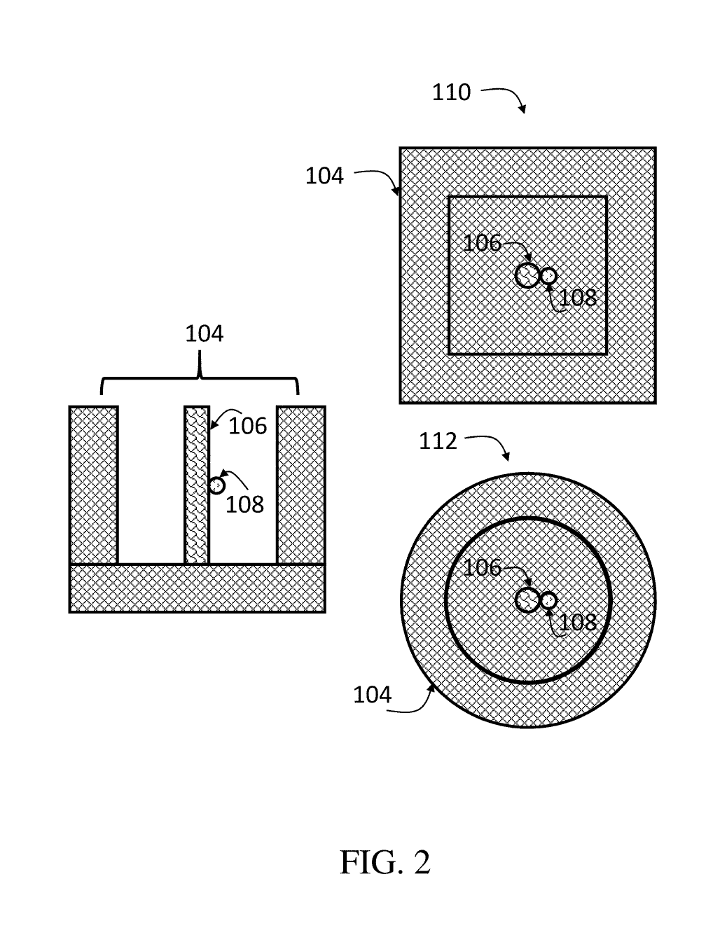

FIG. 2 depicts a side cross-sectional view schematic of a microwell 104 (left), and top cross-sectional view schematics of two alternative configurations 110 and 112 (right) showing the microwell as having a square cross-section (110) or a circular cross-section (112), according to some illustrative embodiments. Other cross-section shapes are also possible. While configurations 110 and 112 depict a circular cross-section of post 106, other cross-section shapes of the post 106 are also possible.

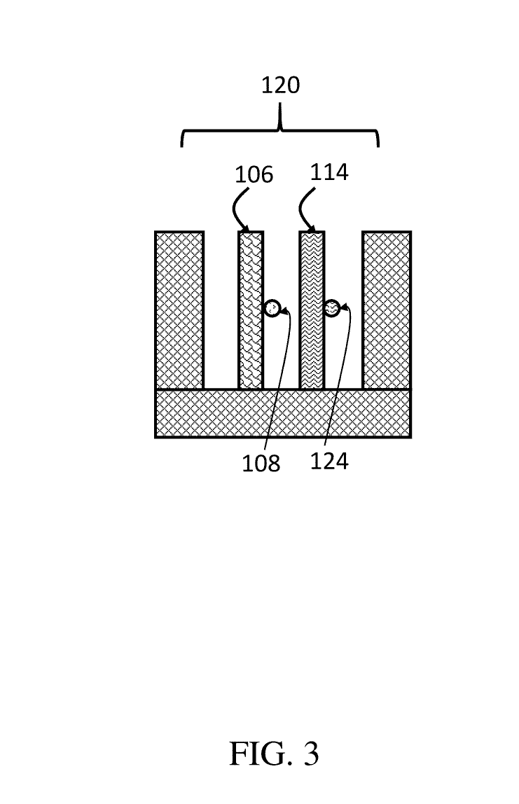

FIG. 3 depicts a side cross-sectional view schematic of a first multiplexed microwell 120 comprising a first post 106 including a first probe 108, and a second post 114 including a second probe 124, according to some illustrative embodiments. In certain embodiments, first probe 108 is chemically different from second probe 124. Second post 114 may comprise the same material as the first post 106 or a different material from the first post 106. Any suitable number of additional posts and corresponding probes (e.g., one, two, three, four) are also possible.

FIG. 4 depicts a side cross-sectional view schematic of an apparatus 200 comprising a first microwell 104 comprising a post 106 including a probe 108, and a substrate 202 comprising second microwells 204, according to some illustrative embodiments. In some embodiments, an apparatus comprises an array of apparatus 200 (e.g., in a similar manner as the array 102 of microwells 104 in FIG. 1). Second microwells 204 may have diameters that are less the diameter of first microwell 104. Second microwells 204 may have spacings 205 between them that are less than the diameter of the first microwell and/or less than the spacings 107 between the first microwell 104 and any other first microwells in an array of first microwells. In some embodiments, a surface 203 of the substrate 202 is proximate (e.g., contacting) a surface 103 of the first microwell 104 such that the contents of the first microwell 104 are physically isolated from any other first microwells 104 (e.g., as in the microwell array of FIG. 1), enclosing a reactor volume 105 (e.g., between or equal to 0.1 nL and 50 microliters, e.g., between or equal to 1 nL and 5 nL, between or equal to 3 nL and 4 nL). Other substrates, and other cross-section shapes and sizes of the first microwell, the post, and the second microwells are also possible.

It should be appreciated that the terms “first”, and “second” microwells, microwell arrays, probes, and posts, as used herein, refer to different microwells, microwell arrays, probes, and posts, within the apparatus, and are not meant to be limiting with respect to the location of that microwell, microwell array, probe, or post. Furthermore, in some embodiments, additional microwells, microwell arrays, probes, and posts (e.g., “third”, “fourth”, “fifth”, “sixth”, or “seventh” microwells, microwell arrays, probes, and posts) may be present in addition to the ones shown in the figures. It should also be appreciated that not all microwells, microwell arrays, probes, and posts shown in the figures need be present in some embodiments.

U.S. Provisional Patent Application Ser. No. 62/585,771, filed Nov. 14, 2017, and entitled “Bioassays from Tissue Sections and Cells Using Functionalized Hydrogels in Isolated Microwell Arrays,” is incorporated herein by reference in its entirety for all purposes.

The following examples are intended to illustrate certain embodiments of the present invention, but do not exemplify the full scope of the invention.

### EXAMPLE

Bioassays from Tissue Sections and Cells Using Functionalized Hydrogels in Isolated Microwell Arrays
a. General Purpose

This example demonstrates a platform that was developed for multiplex and quantitative microRNA (miRNA) measurements in sealed microwell arrays. Each device was capable of performing 100-1000 parallel multiplex assays from raw and fixed cell and tissue samples using simple assay workflows without the need for sample preparation. By controlling the size of the hydrogel-based sensors photopatterned in each isolated microwell, miRNA from ˜10-100 cells per microwell could be measured without the need for signal amplification. The array could be applied to tissue sections to measure spatially resolved miRNA from the tissue.

### Background

Lung cancer is the deadliest cancer worldwide and is a challenge to treat. Despite the availability of targeted and immuno-therapies, many lung cancer patients are intrinsically resistant to treatment or develop drug resistance in a few months. One challenge is the existence of extensive tumor cell heterogeneity and the lack of adequate pathological and biomarker tests for personalized treatment strategies. MicroRNAs (miRNAs) have emerged as sensitive and robust markers for cancer diagnosis and prognosis (with commercially available tests based on miRNA expression available); as well as therapeutics (with miRNA mimic and anti-miRs in clinical trials). There is a technological gap, however, for miRNA probing technologies that can quantitatively assess tumor heterogeneity in a manner that is relevant to pathologists. Here a sensitive miRNA detection and quantification method was developed that can be applied to single cancer cells in an effort to develop a better understanding of lung cancer heterogeneity as well as develop more reliable diagnostic and prognostic tests for lung cancer patients.

Lung cancer: Lung cancer has been the most common cancer since 1985, resulting in 1.59 million deaths worldwide per year. In 2014, it was projected that 224,210 new cases would be diagnosed in the United States alone, resulting in 159,620 deaths. This represented over 25% of all cancers deaths, and 4 times the number of breast cancer deaths in the U.S. In non-small-cell lung carcinoma (NSCLC), a common mechanism of tumor progression is over-expression or tumor-acquired mutation of the EGFR-RAS pathway and loss of wild type function of the tumor suppressive transcription factor p53. While targeted therapies, such as small-molecules antagonizing single proteins in the EGFR, VEGF, RAS, and PI3K pathways have shown encouraging results for subsets of patients, all patients ultimately develop resistance, as these drugs create selective pressure for additional mutations in the tumors.

Tumor heterogeneity and tumor microenvironment: Tumors of many organs, including of the lung, exhibit intra-tumoral heterogeneity, meaning that the individual cells of a tumor can display different gene expression profiles, mutational loads and phenotypes, including stemness. This variability makes it difficult for drug treatment to eliminate the entire tumor and indeed, heterogeneity is altered following treatment suggesting that drugs kill a portion of the cells but some resistant clones survive to grow out, resulting, in some cases, in more aggressive cancers. A better understanding of tumor heterogeneity prior to treatment could be used for more personalized, informed treatments resulting in a better prognosis for patients.

MicroRNAs as cancer therapeutics and diagnostics: MiRNAs are small, non-coding RNAs that regulate gene expression and are involved in multiple biological processes. Thousands of human miRNAs are known and many of these are found mis-expressed in tumors (and other disease tissue) relative to normal tissue. In addition, their stability in bodily fluids has allowed their use as non-invasive biomarkers. In particular, miRNAs have emerged as sensitive and stable biomarkers for cancer diagnosis and prognosis, and importantly because of their tissue specificity, provide better performance and information content than mRNA biomarkers. While miRNA expression levels have been found to be predictive of response to cancer therapies in numerous cancers, including NSCLC, much of this work has focused on a select few cancer-related miRNAs with exceptional promise in NSCLC diagnostics and therapeutics: let-7, miR-34, miR-21 and miR-155. All of these miRNAs map to regions commonly altered in lung cancer, and their altered expression in NSCLC has been shown to be a biomarker for poor outcome. In addition, they act in pathways important for lung cancer progression and metastasis such as EGFR/RAS and p53. For example, let-7 regulates RAS and MYC, while miR-34 regulates MET, BCL2 and multiple cell cycle oncogenes.

b. Technical Description

Many cancer patients develop drug resistance due to extensive tumor cell heterogeneity and the lack of tests for personalized treatment strategies. MiRNAs have emerged as sensitive and stable cancer biomarkers, but traditional miRNA analysis techniques are time-consuming, lack either multiplexing or throughput, and have clinically impractical assay workflows. Consequently, there was a technological gap for miRNA probing technologies with simple, translatable workflows that can quantitatively resolve tumor heterogeneity. To address this gap, a miRNA quantification platform was developed with the capacity to perform parallel, multiplex, and sensitive miRNA measurements in isolated microwells that can be applied to raw cancer cells with no prior sample preparation (e.g., nucleic acid extraction).

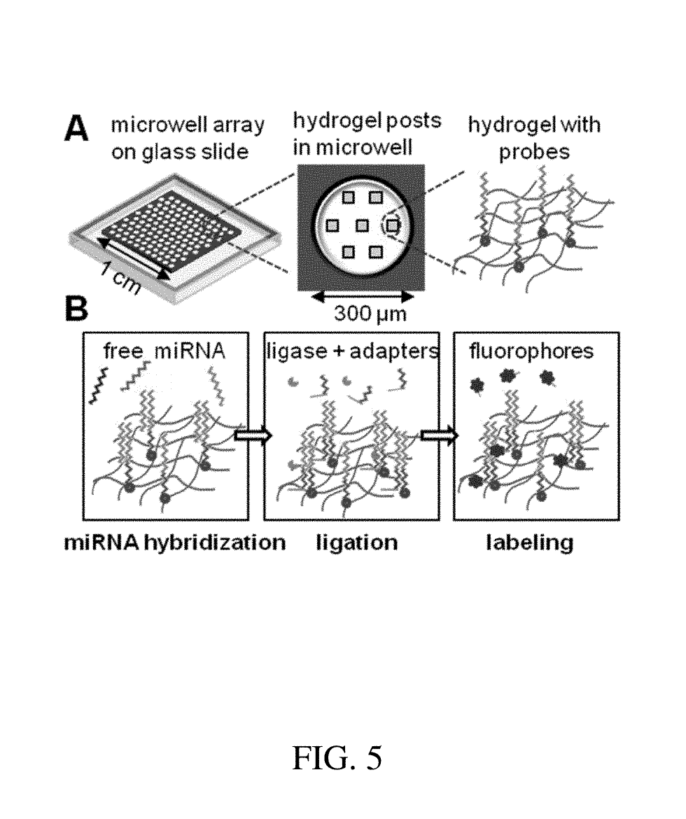

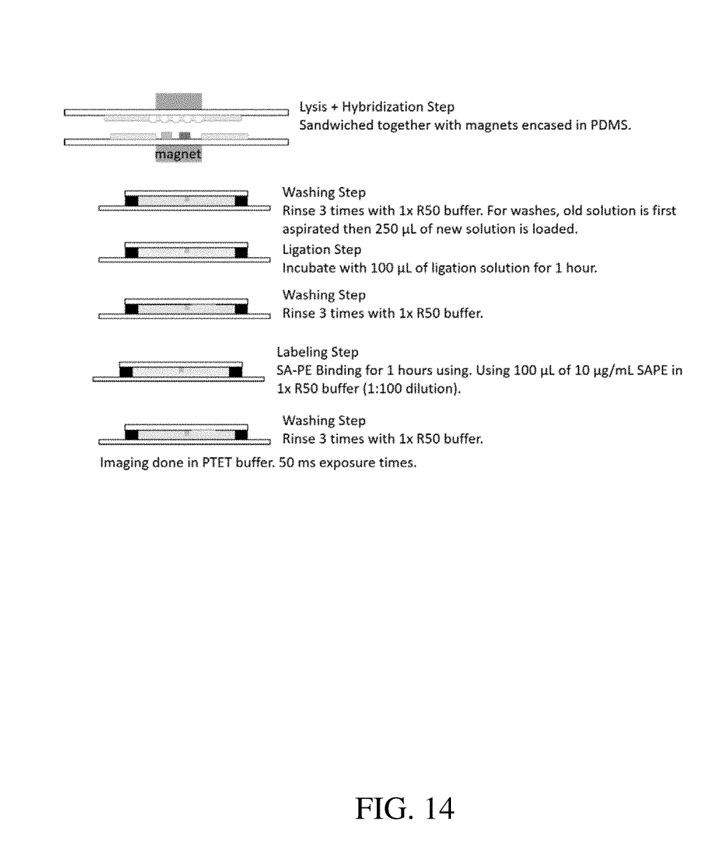

The use of hydrogel particles for multiplex miRNA assays from raw cell samples has been demonstrated. While robust, this particle-based method requires separate 100 μL containers for parallel measurements, each with ˜1000 cells to measure miRNA content. Given these limitations, a microwell array-based approach was developed that is capable of performing 100-1000 parallel assays on a single device while retaining the capacity for multiplex measurements, simple assay workflows (e.g., FIG. 5, FIG. 14), and no need for sample preparation (e.g., FIG. 6).

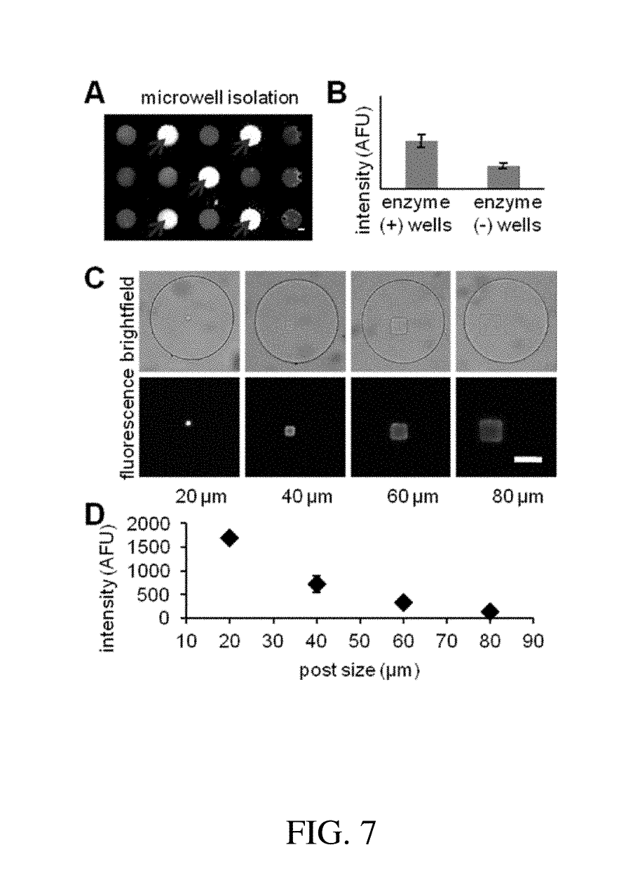

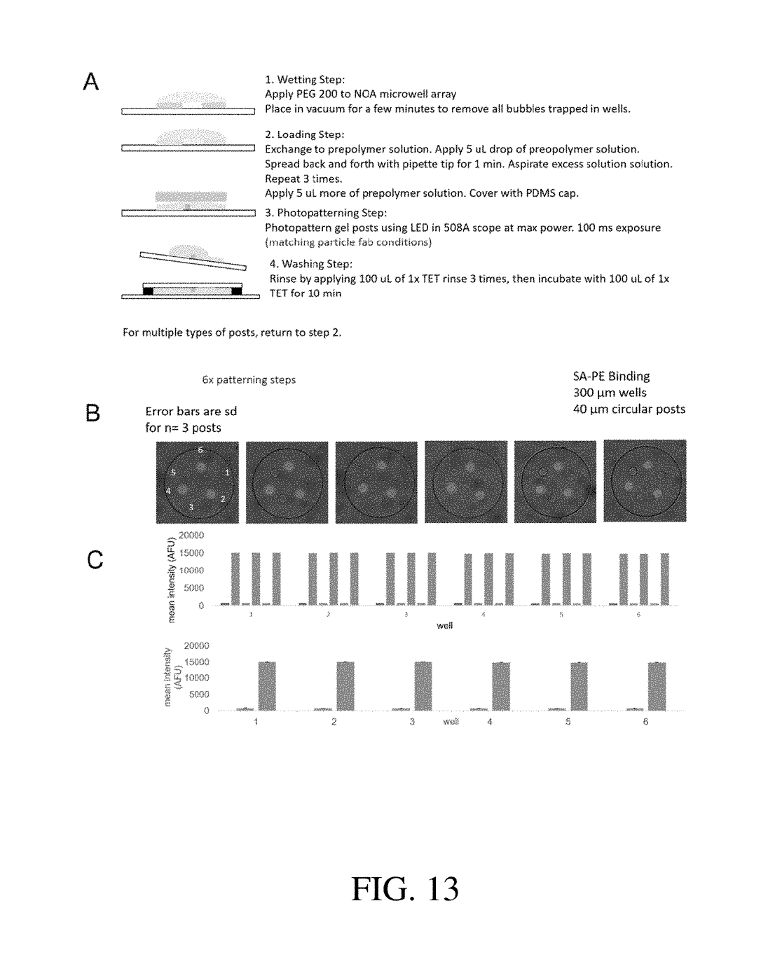

Microwell arrays were formed from photocured Norland Optical Adhesive 81 (NOA81) on acrylated glass slides using PDMS molds. Polyethylene glycol diacrylate (PEGDA) microposts functionalized with DNA probes complimentary to specific miRNA were photopatterned and attached to the glass surface inside the microwells. Microposts with probes complimentary to different miRNAs were photopatterned in the same microwell by sequential exchanges of the prepolymer solution (e.g., FIG. 6B, FIG. 13). By using magnets to seal together sandwiches of microwell arrays, lysis and hybridization agents were delivered to the array containing cells and functional hydrogels (e.g., FIG. 6A) while retaining each separate well isolated (e.g., FIG. 7A-B).

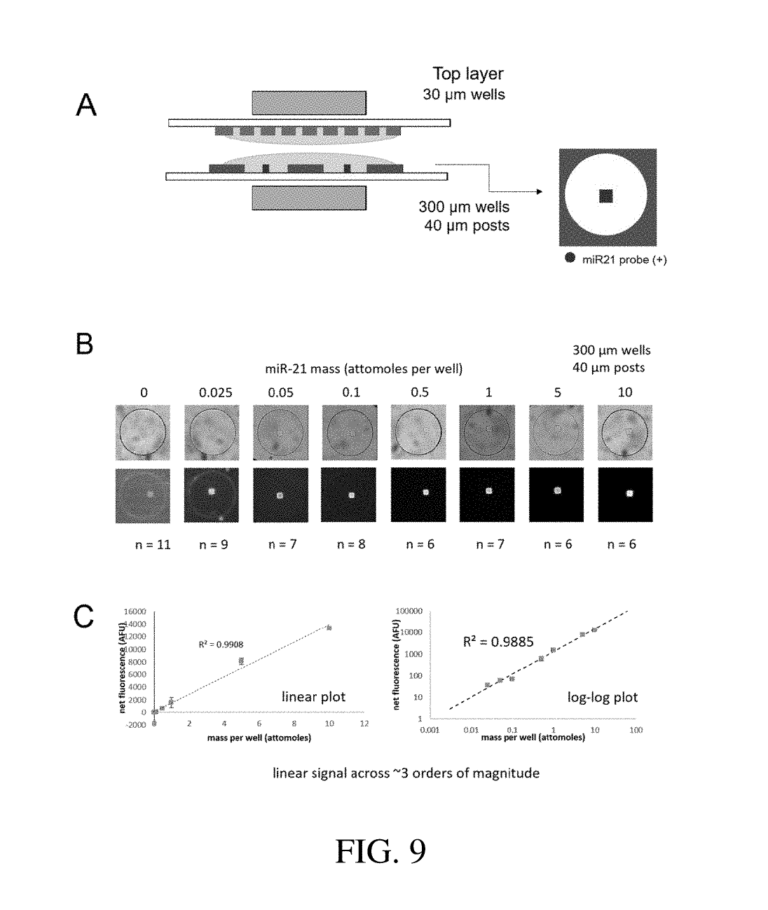

By reducing the total sensing surface (from ˜50 particles in a tube to individual size-tunable microposts), miRNA samples were analyzed that were ˜100× lower in miRNA mass without the need for signal amplification (e.g., FIG. 7, FIG. 9). Specifically, detection of miR-21 from ˜10-100 Calu-6 cells per well was shown (e.g., FIG. 6C-D). By reducing the photopatterned micropost sizes, assay sensitivity was further enhanced without signal amplification (e.g., FIGS. 7C-D).

The cell assay described above was adapted to facilitate miRNA profiling of a field of cells, such as a slice through a tissue section from a tumor sample. A microwell array containing immobilized miRNA probes and detergents for cell lysis was applied directly to a specimen of interest (e.g., FIG. 10). The cells in the section were lysed by diffusive introduction of detergents contained in the microwell array. miRNA in turn diffused primarily into the gel in the adjacent wells for local capture—thus preserving their relative spatial location.

FIG. 1A is a schematic diagram of an apparatus comprising a microwell array, in which each microwell contains microposts (posts having a diameter on the order of from 1 to 100 microns) functionalized with corresponding microRNA capture probes, chemically different from the probes on a different micropost in the same microwell. FIG. 5B is a schematic of a method of assaying miRNAs using hybridization with a probe, ligation of an adapter to hybridized probe-miRNA pairs using a ligase, and labeling of probe-miRNA pairs using fluorophores configured to interact with the adapter.

FIG. 6A is a schematic of a method of assaying miRNAs in microwells, involving: (1) cell settling into microwells having posts; (2) delivering lysis buffer to the microwells using a substrate comprising smaller microwells and sealing (also referred to herein as physically isolating or physically separating) each larger microwell from every other larger microwell using the substrate by positioning a surface of the substrate proximate a surface of the larger microwell array; (3) cell lysis and hybridization of miRNA from the lysed cells with a probe on a post in a microwell; (4) washing the larger microwell array, ligation of an adapter to hybridized probe-miRNA pairs using a ligase, and labeling of probe-miRNA pairs using fluorophores configured to interact with the adapter.

FIG. 6B is a composite fluorescence and brightfield image of an miR-21 assay using sealed microwells. The post having the miR-21 probe (+) fluoresced after following the protocol of FIG. 6A, whereas the post without the probe (−) did not. Scale bars are 100 μm (microns).

FIG. 6C shows brightfield images of Calu-6 cells after settling (top right) and fluorescence images after miR-21 assay method (bottom right) in comparison with no cells (left). Scale bars are 100 μm (microns).

FIG. 6D shows plots of average (n=18; top) and individual (bottom) net fluorescence from posts in microwells in an miR-21 assay. Net signal was the mean fluorescence intensity difference between probe and no probe posts. As the number of cells per well increased, the net fluorescence increased.

FIG. 7A is a fluorescence micrograph of an enzymatic reaction (between an analyte and a probe) in microwells 1 hr after sealing with a substrate comprising smaller microwells. Arrows indicate microwells with enzyme-functionalized posts. Scale bar is 100 μm (microns).

FIG. 7B is a fluorescence plot of the enzymatic reaction of FIG. 7A.

FIG. 7C shows brightfield (top) and fluorescence (bottom) images of microposts of different sizes after a miR-21 assay in sealed microwells (0.5 attomoles of miR-21 per well). Scale bar is 100 μm (microns).

FIG. 7D shows net mean fluorescence intensity plots of microposts of different sizes after the miR-21 assay of FIG. 7C in sealed microwells (0.5 attomoles of miR-21 per well) (n=3).

FIGS. 9A-C demonstrate an illustrative miRNA quantitation. FIG. 9A is a schematic of an assay for miRNA (e.g., miR-21) similar to FIG. 6A, in accordance with certain embodiments. The microwells in which cells settled were 300 microns in diameter, and posts (probe articles) were 40 microns in diameter. The smaller microwells of the substrate wetted with lysis buffer and/or hybridization agent(s) were 30 microns in diameter.

FIG. 9B shows brightfield images (top) and fluorescence micrographs (bottom) of hydrogel posts in microwells after assays done with different miR-21 “mass” (attomoles per well). “n” was the number of samples from which a representative image was selected in FIG. 9B.

FIG. 9C shows plots of net fluorescence from posts having miR-21 probes vs. mass per well of miR-21. The plots of FIG. 9C display a positive linear relationship of signal to mass. The extrapolated lower limit of detection is from 100 to 500 times lower than for previous assays done with particles in tubes.

FIGS. 10A-D demonstrate an illustrative miRNA tissue section assay. FIG. 10A is a schematic of an miRNA (e.g., miR-21) tissue section assay using a microwell array with posts, in accordance with certain embodiments.

FIG. 10B shows brightfield and fluorescence composite images of microwells after a miRNA (e.g., miR-21) assay of FIG. 10A, in accordance with certain embodiments.

FIG. 10C shows column graphs showing fluorescence intensity signal from each well in a microwell array, in an miR-21 tissue section assay as in FIG. 10A, according to certain embodiments. Variation in intensity indicates spatially resolved tissue heterogeneity with respect to miR-21 amounts.

FIG. 10D shows a heat map of fluorescence signal from a tissue section as analyzed in a microwell array in an miR-21 assay as in FIG. 10A, in accordance with certain embodiments. Variation in intensity indicates spatially resolved tissue heterogeneity with respect to miR-21 amounts.

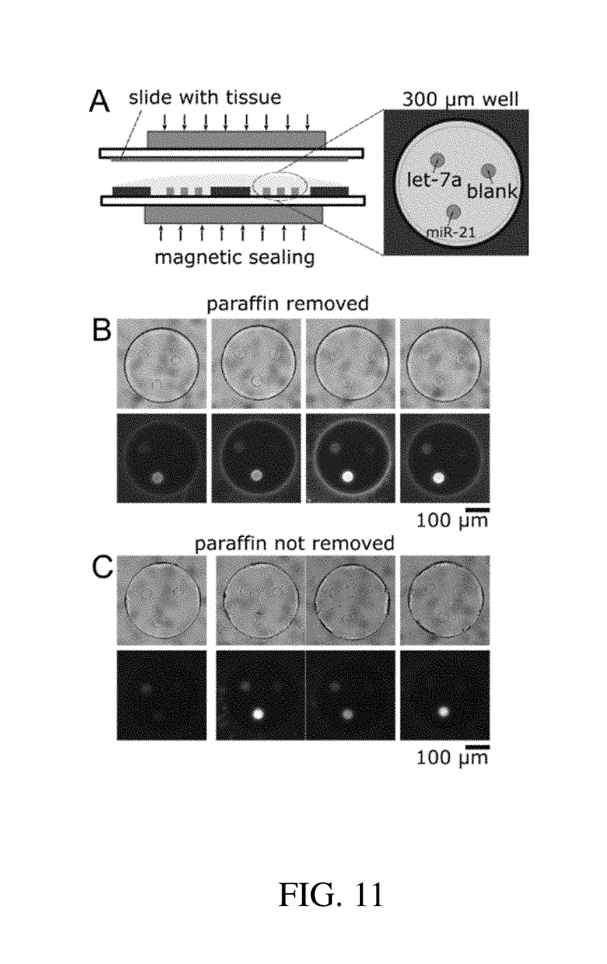

FIGS. 11A-C show multiplex miRNA profiling from fixed tissue sections. FIG. 11A shows an experimental setup schematic; an array with wells containing SDS and proteinase K was directly applied to fixed tissue sections and magnetically sealed for miRNA extraction and hybridization. Wells contained a blank post and posts functionalized with probes complementary to miR-21 and let-7a. FIGS. 11B-C show multiplex miRNA assays from fixed tissue sections of A549 mouse xenograft tumor using the well array. Brightfield (top) and fluorescence (bottom) images of wells following assay with fixed tissue (FIG. 11B) with paraffin removed and (FIG. 11C) without paraffin removal. miRNA was able to be measured directly from fixed tissue sections using a microwell array, further demonstrating the non-fouling nature of the PEG hydrogel posts.

FIGS. 13A-C demonstrate an illustrative gel post fabrication. FIG. 13A is a schematic of a method of gel post fabrication.

FIG. 13B shows brightfield and fluorescence composite images of biotinylated and blank gel posts, fabricated in alternating steps, after streptavidin, r-phycoerythrin conjugate (SA-PE) binding.

FIG. 13C shows plots of mean fluorescence intensities for each post (top) and mean values for biotinylated and blank posts (bottom).

FIG. 14 is a schematic of an assay protocol for a nucleic acid analyte, using a microwell comprising a post having a probe for the analyte, in accordance with certain embodiments.

### Methods

Well Array Fabrication

Polyethylene glycol diacrylate (PEGDA) hydrogel posts were covalently attached to glass substrates using methacrylate-terminated silane monolayer formation. Plain glass microscope slides (Thermo Fisher) were cut into desired dimensions using a diamond scribe (Ted Pella) and Running and Nipping Pliers (Fletcher-Terry) and stored under vacuum until usage. Polydimethylsiloxane (PDMS, Sylgard® 184, Dow Corning) molds were made using standard soft-lithography protocols by mixing elastomer base and curing agent in a 10:1 ratio and cured on a SU-8 (MicroChem) master that was prepared using standard photolithography protocols. Molds were designed to create 1×1 cm arrays of wells with diameters of 300 μm and 30 μm and depths of 35 μm and 38.6 μm, respectively. The arrays contained indexing marks in place of some wells. The individual 1×1 cm PDMS molds were cut using a scalpel and had 1.5 mm inlets punched out (Biopsy Punch, Miltex). Norland Optical Adhesive 81 (NOA81, Thorlabs) well arrays were formed on the acrylated glass slides by filling the PDMS molds using degas-driven flow, UV curing the NOA81, and removing the PDMS molds.

Hydrogel Post Fabrication

Functionalized PEGDA posts were photopolymerized in the nanoliter wells using projection lithography methods. Prepolymer solution containing 18% (v/v) PEGDA 700, 36% (v/v) PEG 200, 4.5% (v/v) Darocur® 1173 photoinitiator (2-hydroxy-2-methylpropiophenone), ˜1× TE buffer, and DNA probes was loaded into the NOA81 300 μm well array. The well array containing prepolymer solution was then covered with a 1-2 mm flat PDMS film. PEGDA posts were then photopolymerized via projection lithography using mylar transparency masks (Fineline) placed in the field-stop slider between a 365 nm UV LED (Thorlabs) and a 20× EC Plan NeoFluor objective (Zeiss) on a Zeiss AX10 inverted fluorescence microscope. Intensities of 720 mW cm⁻² and exposure times of 100-200 ms were used to fabricate circular or square 20-200 μm posts, as specified. Following post photopolymerization, the PDMS film was removed and devices were rinsed with 1× TE buffer with 0.05% (v/v) Tween® 20 (1× TET). Following iterative prepolymer solution loading, post photopolymerization, and wash steps, posts containing different DNA probes were formed within the same well. Devices with functional posts were stored at 4° C. in 1× TET until usage. Hydrogel posts were treated with 500 μM potassium permanganate (Sigma) to oxidize hydrophobic, non-reacted acrylate groups to reduce non-specific binding, as specified.

Cell Samples and Cell Handling

Human lung cancer cell line Calu-6 cells were cultured in Dulbecco's Modified Eagle Medium (high glucose, GIBCO) with 10% fetal bovine serum, 2 mM L-glutamine, and 1% penicillin-streptomycin. Upon reaching 70% confluence, the cells were treated with 0.25% trypsin-EDTA (Gibco) and then frozen with 10% dimethyl sulfoxide (DMSO) (Sigma) in complete culture medium. Frozen cells were kept in liquid nitrogen for long term storage and −80° C. freezer for short term storage before use. Frozen cells were thawed to remove DMSO and were reconstituted into room temperature media before use. The density of cell suspensions were counted using a Bright-Line™ Hemocytometer (Sigma). Immediately preceding experiments with cells, cells were pelleted and resuspended in settling buffer (1× TE, 137 mM NaCl) at the desired densities. A 5 μL drop of the cell suspension was then applied onto the well array devices and cells were allowed to passively settle for 10 min. Devices were then imaged before analysis in order to count the number of cells settled into each well.

miRNA Hybridization Assay

For the hybridization step, the 300 μm well array was sealed against a 30 μm well array using 1.2×0.16 cm disk-shaped neodymium magnets (Grainger). Stacks of 3 magnets were placed on each side of the sandwich. The hybridization buffer inside the wells contained 1× TE, 0.05% (v/v) Tween® 20, and 350 mM NaCl. For synthetic miRNA assays, the hybridization solution contained target microRNA. For cell assays, the hybridization buffer contained ˜2% sodium dodecyl sulfate (SDS) and ˜15 U/mL of proteinase K for cell lysis and miRNA extraction. The hybridization step was done for 90 min at 55° C. (VortTemp™ 56, Labnet). Following hybridization, the magnets were removed and the device was rinsed with 1× TE, 0.05% (v/v) Tween® 20, and 50 mM NaCl (R50) by placing the well array slide face down over a glass slide with ˜500 μm spacers and subsequent solution loading and aspiration steps. Then, the ligation step was done by loading ligation buffer containing the biotinylated linker and T4 DNA ligase and incubating for 1 hour at room temperature. Following ligation, the well array was rinsed with R50 and labeling was done by loading R50 buffer containing 10 μg/mL streptavidin-R-phycoerythrin (SA-PE, Invitrogen) and incubating for 1 hour at room temperature. Devices were then rinsed with R50 to ensure removal of unbound SA-PE before imaging.

Imaging and Data Analysis

Brightfield and fluorescence imaging was done using a Zeiss Axio Observer A1 inverted microscope equipped with a X-Cite 120LED light source (Lumen), 5×, 10×, and 20× EC Plan NeoFluor objectives (Zeiss), and an Andor Clara CCD camera. Images were captured using 100% intensity with 50 ms exposures and no binning in Andor Solis software. SA-PE and FDG imaging was done using XF101-2 (λₑₓ/λₑₘ=525/565 nm) and XF100-3 (λₑₓ/λₑₘ=470/545 nm) filter sets (Omega), respectively. Image analysis was done custom ImageJ (National Institutes of Health) and MATLAB (Mathworks) scripts written in-house.

Well Isolation

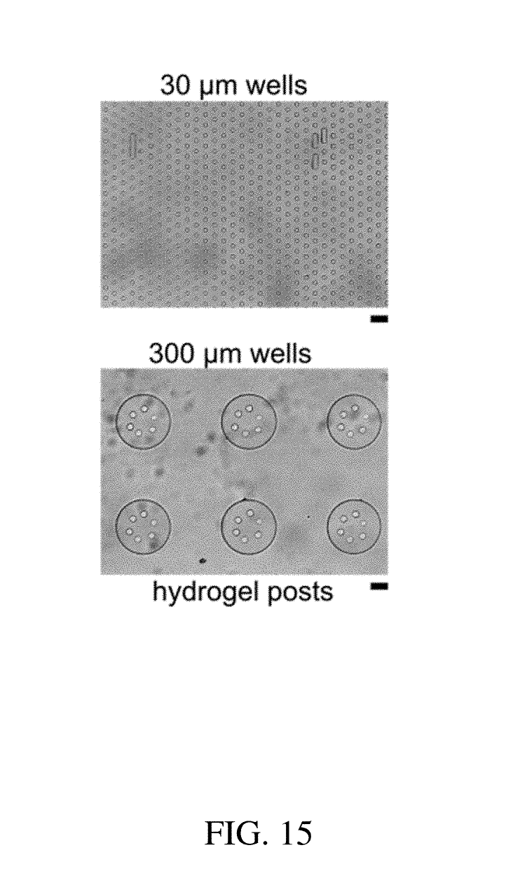

The miRNA assays were performed in devices consisting of well arrays made of NOA81 formed on glass slide substrates (e.g., FIG. 5A). The devices were comprised of two separate layers that were sandwiched together during the miRNA hybridization step to form isolated reactors (e.g., FIG. 6A). Wells with 30 μm diameters were chosen for the top layer to ensure no overlap between reactors when the bottom layer well spacing was 30 μm. When the top array with 30 μm wells was applied onto the 300 μm well array without any alignment, on average 27.5 30 μm wells interfaced with each 300 μm well. Using the geometries of both wells arrays, the volume of each reactor in the sealed microarray sandwich was ˜3.2 nL. There was no overlap between each reactor because the spacing between each 300 μm is greater than or equal to the spacing between each 30 μm well (e.g., FIG. 15). FIG. 15 shows bright field images of a first microwell array having 30 micron diameter microwells (top) and a second microwell array having 300 micron diameter microwells (bottom) having 6 probe-containing posts each per microwell. Scale bar is 100 microns. In some embodiments, the first microwell array and second microwell array come together to form an apparatus. By performing the miRNA hybridization assay in wells instead of a centrifuge tube (50 μL) the volume of each reaction was reduced by over four orders of magnitude. PEGDA posts functionalized with DNA probes complimentary to specific miRNA targets were photopolymerized within the 300 μm wells and covalently attached to the acrylated glass substrate (e.g., FIG. 15 bottom). The hybridization step (where free miRNA binds to complimentary DNA probes in the PEGDA posts; e.g., FIG. 5B, hybridization) was performed in the sandwiched configuration. Following hybridization, the sandwich was opened and subsequent steps were performed by incubating the 300 μm array in the specified solutions. During ligation, biotinylated linkers were ligated to the captured miRNA targets (e.g., FIG. 5B, ligation). SA-PE was then introduced which binds to the biotinylated linkers and fluorescently label the captured targets (e.g., FIG. 5B, labeling).

In order to determine if the ˜3.2 nL reactors were properly isolated from each other during the hybridization step, a fluorometric assay was performed in the magnetically sealed well array sandwiches (e.g., FIG. 7A). 200 μm circular posts containing biotinylated DNA probes were photopolymerized in select 300 μm wells. Streptavidin-β-galactosidase conjugates (SAB, Invitrogen) were then bound to the DNA probes. The 300 μm well arrays were then sealed against a 30 μm well array containing fluorescein di-β-D-galactopyranoside (FDG, Thermo Fisher) substrate. The sandwich was then incubated for 1 hour at room temperature before imaging. High viscosity ethyl cyanoacrylate adhesive (World Precision Instruments) was applied around the edges of the glass slides of array sandwich in order keep the device sealed after the removal of the magnets for imaging. The fluorescence intensity of each well was measured using the average intensity in 100 pixel (246 μm) diameter circular windows from images collected using a 5× objective. The wells with enzyme functionalized posts had mean intensities of 5090±680 AFU (n=5 wells) while the wells without enzyme functionalized posts had mean intensities of 2490±310 AFU (n=10 wells). ±values indicate standard deviation (SD). The brighter fluorescence signal was observed only in wells that contained the enzyme-functionalized posts, indicating that the wells were isolated reactors during the timescale of the experiment.

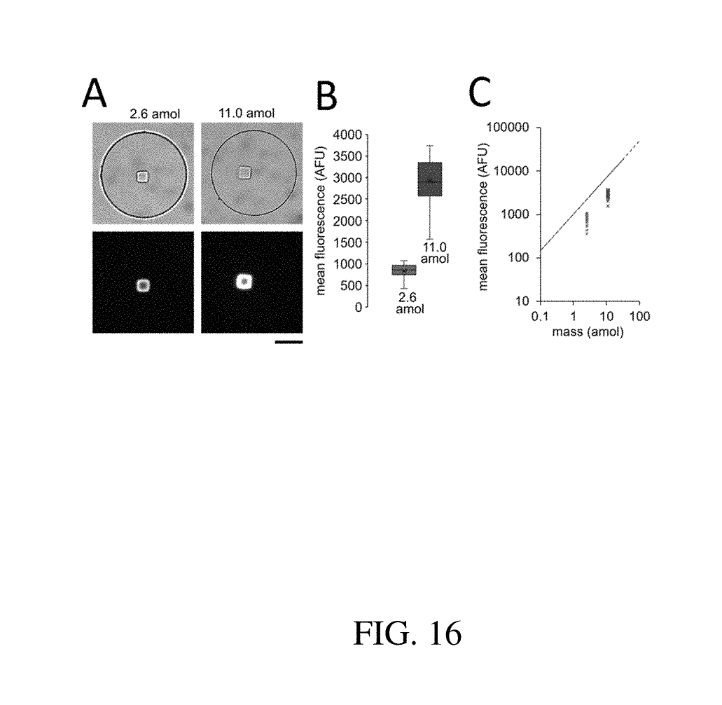

To assess the reproducibility of reagent delivery during the nanoliter reactor assembly process, a SA-PE binding assay was performed on 40 μm PEGDA posts functionalized with biotinylated probes housed inside 300 μm wells sealed against a 30 μm well array (FIG. 16A).

FIGS. 16A-C show an SA-PE binding assay in sealed wells. 2.6 amol of SA-PE was delivered by including 1 μg/mL SA-PE in the 30 μm well array before sealing (left) and 11.0 amol delivered by including 1 μg/mL SA-PE in both the top layer and bottom layer before sealing. FIG. 16A shows brightfield (top) and fluorescence (bottom) micrographs following SA-PE binding assay. Scale bars are 100 μm. FIG. 16B shows box plots showing the distribution of mean fluorescence of posts in each condition. The error bars indicate minimum and maximum of the distribution, the ends of the box are the first and third quartiles, the vertical line in the box is the median, and the x is the mean (n=40 wells for 2.6 amol, n=39 wells for 11.0 amol). FIG. 16C shows a plot showing mean post fluorescence vs. loaded mass for each well. The dashed line represents the theoretical maximum mean fluorescence in a 40 μm post estimated for the mass loaded.

These experiments were also used to determine if the amount of captured SA-PE after the assay was consistent with the amount theoretically delivered to each well. 2.6 amol of SA-PE were delivered by including 1 μg/mL SA-PE in the 30 μm well array before sealing and 11.0 amol of SA-PE was delivered by including 1 μg/mL SA-PE in both the 30 μm and 300 μm well arrays before sealing. For simplicity, the volume occupied by the gel posts was neglected from the volume calculations. After completing the binding assay, the mean fluorescence intensity of posts was measured in wells where SA-PE was included in just the top (30 μm well) array (Iₜₒₚ, n=40 wells from 2 separate devices), and in wells where SA-PE was included in both arrays before assembly (I_{top+bottom}, n=39 wells from 2 separate devices). As expected, values were not normally distributed because the maximum possible SA-PE delivered had an upper bound capped by reactor volume and were thus displayed in box plots (FIG. 16B). Based on the estimated differences in loaded mass, a ratio of I_{top+bottom}/Iₜₒₚ˜4.3 was expected and a ratio of ˜3.5 was observed. In order to compare the resulting fluorescence values to the fluorescence expected from the loaded SA-PE mass, a calibration curve of SA-PE fluorescence was developed. The trend in fluorescence values observed was consistent with the expected values and both mean values fell under the theoretical maximum mean fluorescence possible based on the amount of SA-PE loaded, consistent with well isolation (FIG. 16C). A lower coefficient of variability (CV˜16%) was observed when SA-PE was loaded in both layers before isolation, compared to a CV˜21% when SA-PE was included only in the top layer. This decrease in CV is consistent with variability in delivered mass attributable to geometric factors, as when SA-PE in included in both layers, a smaller fraction of the volume containing SA-PE changes due to variations in well overlap numbers. However, not all of the variability observed in either case can be attributed to geometric factors alone. Given that analytes were quantitatively measured and wells were isolated during the device sandwich assembly, quantitative miRNA assays were next performed in our platforming and a framework for understanding assay performance was developed.

miRNA Binding Assay Performance

Without being bound by theory, a theoretical framework for describing the performance of the hydrogel-based particle miRNA assay is governed by the following equation:

$$I = {F}_{e} * \frac{{{V}_{r} [ {C}_{target} ]}_{0}}{{N}_{p} {A}_{p}} * ( 1 - {e}^{- t \text{/} τ} ) \qquad ( 1 )$$

where I is the net mean fluorescence measured in the hydrogels corresponding to captured miRNA signal at the end of the assay, Fₑ is a fluorescence efficiency constant depending on the fluorophore and imaging parameters, Vᵣ is the reaction volume the assay takes place in, [C_{target}]₀ is the initial concentration of the target analyte in the reaction volume, Nₚ is the number of particles in the reaction volume, Aₚ is the 2D area of each particle measured during imaging, t is the assay hybridization time, and τ is a time constant describing the timescale of capturing all of the targets in the reaction volume. Thus, when t>>τ, one can assume that approximately all of the target mass (mass_{target}=Vᵣ*[C_{target}]₀) is captured and the maximum fluorescence for that device geometry and setup is achieved:

$${I}_{max} = {F}_{e} * \frac{{mass}_{target}}{{N}_{p} {A}_{p}} \qquad ( 2 )$$

Without being bound by theory, due to the high porosity of the PEGDA structure and large amount of DNA probes incorporated in the hydrogels, analyte binding was fast compared to analyte diffusion within the hydrogel structure (Damköhler number (Dₐ)>>1), thus τ was dominated by mass transport. This effect manifested itself experimentally as the appearance of a boundary layer of brighter fluorescence at the edges of the hydrogel sensing surface, as targets (also referred to herein as analytes)were captured in that boundary layer before they were able to diffusively penetrate further into the hydrogel structure. In a previous particle-based assay Vᵣ˜50 μL and hybridization times t˜90 min, thus τ was minimized by using vigorous convective mixing to ensure efficient transport of the targets to the hydrogel particle sensing surfaces and also by loading Nₚ˜50 particles per reaction to increase total sensing surface. However, equation (2) indicates that increasing Nₚ lowers Iₘₐₓ, meaning there is a tradeoff in assay sensitivity and assay time. In other words, increasing total sensing area (Aₜₒₜₐₗ=Nₚ*Aₚ) resulted in faster capture of the available targets, but lowered the fluorescence signal per area measured upon completion of the assay.

In the nanoliter well-based assay presented here, Vᵣ was reduced from 50 μL to 3.2 nL by miniaturization of each reaction volume. Without being bound by theory, using just diffusive mass transport of miRNA targets to a single 40 μm hydrogel post in a 300 μm well it was estimated that τ<6 min, and thus t>>τ for a hybridization time of t=90 min. Thus, the reduction of Vᵣ facilitated a reduction in Aₜₒₜₐₗ without needing to increase assay hybridization time t. In addition to reducing Nₚ from 50 to 1, the hydrogel area Aₚ was also reduced without any challenges in handling because the posts were covalently attached within the wells. Therefore, by reducing Aₜₒₜₐₗ by a factor of ˜100, this theoretical framework predicted that the well-based assay should be ˜100× more sensitive (detection of 100× less mass_{target} in a given reactor) compared to the particle-based assay without using any signal amplification. For the well-based assay, I≈Iₘₐₓ and therefore assay performance was described by:

$$I = {F}_{e} * \frac{{mass}_{target}}{{N}_{p} {A}_{p}} = {F}_{e} * \frac{{mass}_{target}}{{A}_{total}} \qquad ( 3 )$$

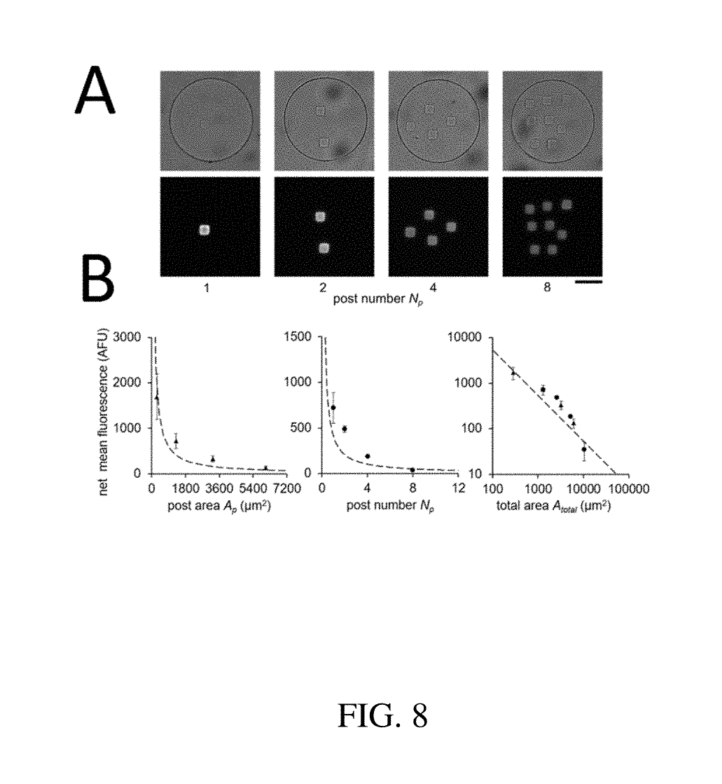

In order to find support for the theoretical framework developed above, miR-21 assays were performed in isolated wells with different number of posts and posts of different sizes with DNA probes complementary to miR-21. miR-21 has been shown to be upregulated in non-small cell lung cancers and has potential as a biomarker for patient outcome. During sandwich assembly, 0.5 amol of miR-21 was delivered to each nanoliter reactor. Wells contained a single square post of varying area (e.g., FIG. 7C; 300, 1300, 3200, and 6100 microns squared, respectively) or varying numbers of posts (e.g., FIG. 8A). Using this approach, post area (Aₚ) and the number of posts (Nₚ) was varied independently. The net mean signal of each post decreased with increasing post area, as well as with increasing number of posts per well, as expected from equation (3) (e.g., FIG. 8B). In order to determine that the observed differences in mean fluorescence signal were not the result of changes in probe incorporation in posts of different area, a well array was made with posts of different area that contained biotinylated probes in different wells and performed a binding assay by incubating with SA-PE. Because in this configuration the wells were not isolated with an excess of SA-PE molecules in solution, approximately all DNA probes in the hydrogels were labeled with SA-PE. There was no observed statistically significant difference in mean fluorescence signal for posts of different area when the binding assay was done without well isolation, indicating that the method of making did not result in posts of different area having different probe incorporation efficiencies. Therefore, these results indicated that when assays in the well arrays were done with isolated wells, the differences in mean fluorescence signal measured were the result of the same loaded target mass binding to different hydrogel sensing areas.

Without being bound by theory, from equation (3), the model predicts that net mean fluorescence signal I should scale with Aₜₒₜₐₗ⁻¹. By plotting the I measured from the isolated well array miR-21 assays with Aₜₒₜₐₗ as the independent variable, the results were qualitatively consistent with the predicted relationship (e.g., FIG. 8B). Additionally, using an experimentally estimated Fₑ, the results had quantitative agreement with the theoretically expected I values. The model predicted that as long as I≈Iₘₐₓ, minimizing post area (Aₚ) resulted in high assay performance. As expected from theory, the experimental results showed that the posts with the smallest area (Aₚ˜300 μm²) had the highest net mean fluorescence I upon completion of the miR-21 assay (e.g., FIG. 7C, FIG. 8B). Because at least some of these posts had an aspect ratio greater than 1 (post width<20 μm), occasionally toppled posts occurred. Thus, posts with widths ˜40 μm were chosen due to their balance of reliability and performance in 35 μm deep wells.

Having developed an understanding of the assay performance, experiments were performed to estimate a lower limit of detection (LLOD). By delivering 0.5 amol of miR-21 to each isolated nanoliter reactor (e.g., FIG. 7C, FIGS. 8A-B), miRNA assays were demonstrated to be able to detect lower miRNA masses per reactor compared to previously demonstrated particle-based assays, which have an estimated LLOD of ˜2-5 amol, without signal amplification. By varying the amount of miRNA mass delivered to different nanoliter well reactors (each with a single square post), a calibration curve was constructed to determine the quantitative dynamic range and LLOD of the assay. 0, 0.025, 0.05, 0.1, 0.5, 1, 5, and 10 amol of miR-21 was delivered to each well in different devices and net mean fluorescence of posts was measured for each condition. (e.g., FIG. 9B). As expected from equation (3), as mass_{target} per well decreased, the measured net mean fluorescence decreased linearly (e.g., FIG. 9C, measured in arbitrary fluorescence units (AFU)), which allowed the assay to perform quantification of unknown analyte quantities. The net mean fluorescence showed this linear relationship with loaded mass for over 3 orders of magnitude with R²=0.99. Using these results, the concentration at which signal over noise (SNR) is equal to 3 was extrapolated, and an LLOD of 0.004 amol was estimated for our assay. As expected from the theoretical model, the LLOD was >100× lower than previously reported for hydrogel particle-based assays done without signal amplification. These results demonstrated the ability to perform sensitive and quantitative miRNA assays across a large dynamic range. Because multiplexing capabilities can be important for translational miRNA assays, a wide dynamic range may be advantageous given that expression levels vary across orders of magnitude for different miRNA targets. Enhanced sensitivity facilitates analysis of miRNA targets with low expression levels and from precious, material-limited specimens.

Multiplex miRNA Assays

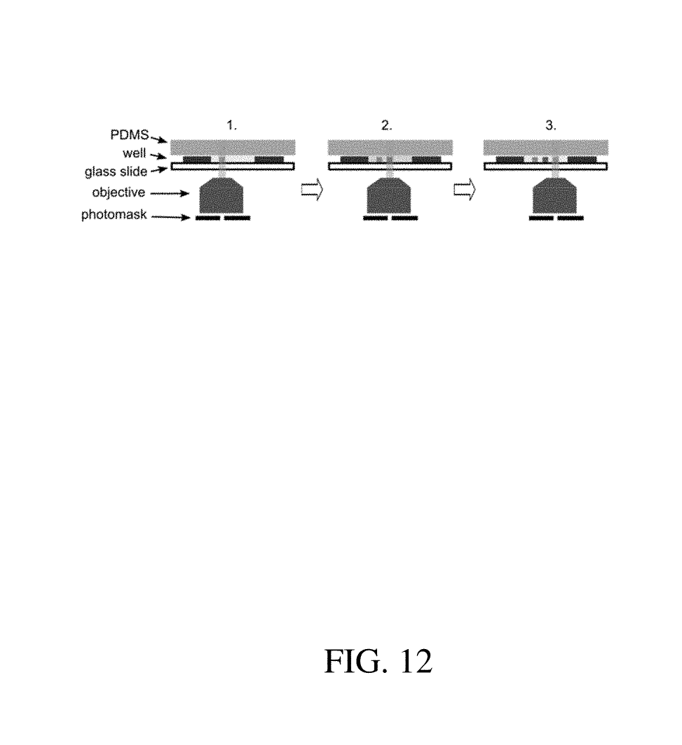

In order to make multiplex miRNA hybridization assays, different PEGDA posts were formed, each functionalized with DNA probes complimentary to different miRNA targets within a single well. To form posts functionalized with different DNA probes within a single well, alternating prepolymer solution loading and exposure steps were performed (e.g., FIG. 12). FIG. 12 shows an example of post fabrication for different posts within a single well, including a post fabrication schematic. Steps 1-3 show photopolymerization of 3 posts within a well with alternating functionalization. Step 1: Load prepolymer solution for post type 1 (e.g., blank posts), photopolymerize first post. Step 2: Exchange to prepolymer solution for post type 2 (e.g., biotinylated posts), photopolymerize second post. Step 3: Exchange to prepolymer solution for post type 3 (e.g., blank posts), photopolymerize third post. In order to evaluate the fabrication reproducibility of this approach and assess proper prepolymer solution loading and exchange, circular PEGDA posts were photopolymerized with or without biotinylated DNA probes in alternating steps. The loading and exposure steps were done 6 times resulting in each well containing 6 posts (3 for each condition). Circular posts were used in order to facilitate alignment of multiple posts relative to each other. After fabrication, a SA-PE binding assay was performed by incubating the device in a solution containing SA-PE (e.g., FIG. 13B). The mean fluorescence signal of each post following the binding assay completion was measured using a circular windows of 40 pixels (24 μm) placed over each post (e.g., FIG. 13C), resulting in an average coefficient of variation (CV) of the net mean fluorescence between different wells of 0.3±0.1% (3 posts of each type per well, n=6 wells). Within each well, the fluorescence signal from blank posts had an average CV of 1.9±0.8% (3 posts per well, n=6 wells), and the fluorescence signal from biotinylated posts had an average CV 0.3±0.1% (3 posts per well, n=6 wells). These results showed that fabrication of posts at different positions within wells and across different wells is reproducible and that the protocol achieved proper prepolymer solution exchange between each post fabrication step.

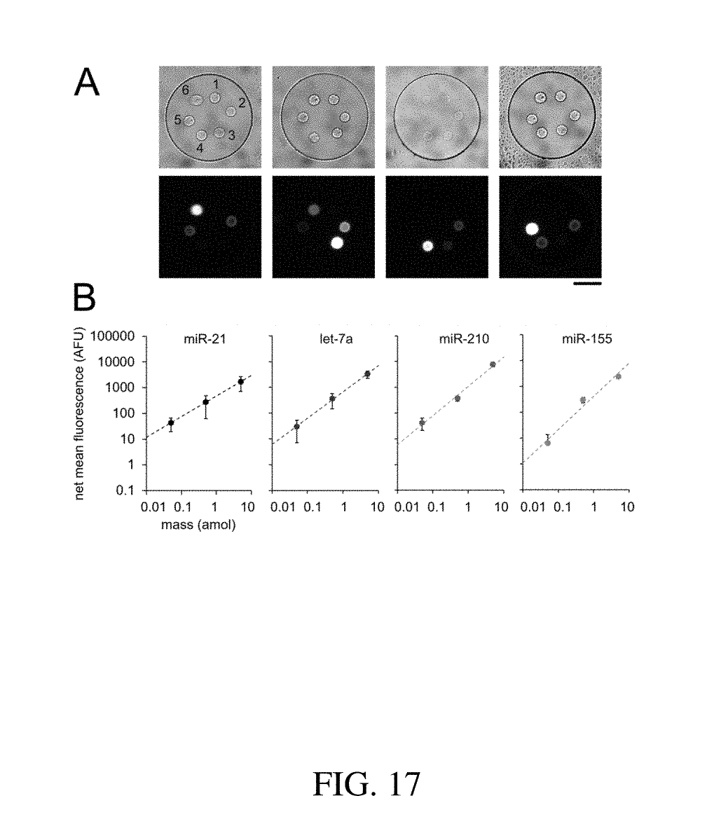

Having demonstrated the ability to reproducibly fabricate differently functionalized posts within separate wells, next multiplex miRNA assays were performed. We used the same fabrication protocol to make devices with circular PEGDA posts with probes complimentary to 6 different miRNA targets (cel-miR-238, cel-miR-54, miR-21, let-7a, miR-210, miR-155) within a given well (e.g., FIG. 17A).

FIGS. 17A-B show multiplexed miRNA assays in sealed nanoliter wells. FIG. 17A shows brightfield (top) and fluorescence (bottom) micrographs following multiplex miRNA hybridization assay in sealed wells. The circular posts contained DNA probes complementary to (1) cel-miR-238, (2) cel-miR-54, (3) miR-21, (4) let-7a, (5) miR-210, and (6) miR-155, as labeled. Scale bar is 100 μm. FIG. 17B shows plots showing net mean fluorescence for different miRNAs as a function of loaded mass per well. cel-miR-238 was used as a negative control and cel-miR-54 was used as a positive loading control. Error bars represent ±SD (n≥4 wells for each condition). Dashed lines indicate linear fit, R²=1.00 for all conditions.

Panels of 3 to 7 miRNAs have been demonstrated for targeted profiling assays of lung cancer and other diseases. let-7a, miR-210, and miR-155 have been shown to be dysregulated in cancer. cel-miR-238 and cel-miR-54 are expressed in C. elegans. miRNA assays were performed in isolated well devices, each with different amounts of the different miRNA targets. Cel-miR-238 was used as a negative control and kept at 0 amol per well for all assays. Cel-miR-54 was used as a loading control and kept at 0.5 amol per well for all assays. miR-21, let-7a, miR-210, and miR-155 amounts were varied between 0, 0.05, 0.5, and 5 amol in the different assays. Using the internal negative and positive controls as loading controls, linear calibration curves were obtained for all 4 miRNA targets that varied in mass per well, with R² values of ˜1 for all for targets. (e.g., FIG. 17B). These results demonstrated the capacity of this platform for quantitative, multiplex assays. The multiplexing scheme used here involves spatial separation of the different posts and therefore, multiplexing is limited by the number of posts that can be made within a given well. Using 20 μm posts (FIG. 7A) with 20 μm spacing, ˜50 posts can be fabricated within a 300 μm single well. If even higher multiplexing were needed, biotinylated adapters with different chemistries could be used to attach different fluorophores to allow spectral multiplexing within posts functionalized with multiple probes to different targets.

miRNA Assays from Unprocessed Cells

For assays with synthetic miRNA targets, miRNA was delivered to the nanoliter reactors during the well array sandwich assembly. For assays with cells, however, cells were first settled into the bottom layer that contained the PEGDA posts and lysis reagents were delivered to the reactors for miRNA extraction during the well array sandwich assembly. Calu-6 cells were settled into devices with 300 μm wells containing PEGDA posts with probes complementary to miR-21. Cells were suspended to densities of 0.25. 1, 2, and 8 million/mL which resulted in 14±8 (n=37), 53±20 (n=28), 110±19 (n=71), and 200±26 (n=116) cells per well, respectively, after 10 min of settling (n=number of wells). Because cells were passively sedimented into the wells from a 5 μL droplet applied onto the well array surface, varying numbers of wells contained cells depending on the cell suspension density. A top layer with 30 μm wells containing lysis buffer was then applied onto the devices with settled cells and magnetically sealed for the cell lysis, miRNA extraction, and miRNA hybridization step (e.g., FIG. 6A). After 90 min at 55° C., the devices were opened and the ligation and labeling steps were conducted as detailed previously for assays with synthetic targets. The mean intensity of the posts in each well was then determined and the net mean intensity was calculated by subtracting the mean intensity of negative controls calculated from devices in which no cells were settled. The negative control wells had mean signals of 0.5±0.3 AFU (n=18 wells). From a total of 252 wells with cells analyzed, 27 wells had net mean miR-21 signal<0. This corresponds to ˜57% of wells with ≤30 cells (24 out of 42 wells), ˜18% of wells with 30<cells≤60 (2 out of 11 wells), ˜5% of wells with 60<cells≤90 (1 out of 19 wells), and 0% of wells with >90 cells. For wells showing net positive signal, the net mean intensity of miR-21 correlated with the number of cells per well (R²=0.45) (e.g., FIG. 6D). Using the linear fit, a LLOD ˜16 cells/well was estimated. As detailed previously, miniaturization of the reaction volumes from 50 μL to 3.2 nL along with reducing the PEGDA sensing surface enhanced our previously demonstrated sensitivity, in this case from ˜1000 cells, down to ˜16 cells. With the demonstrated sensitivity, our platform enables high-throughput analysis of specimens with small cells numbers, such as 3D spheroids, circulating cell clusters organoids, early stage embryos, small whole organisms, and precious, material-limited biopsies. Additionally, instead of relying only on passive sedimentation, cells may be captured and even selected by implementing chemistries that bind to cell surface markers within wells.

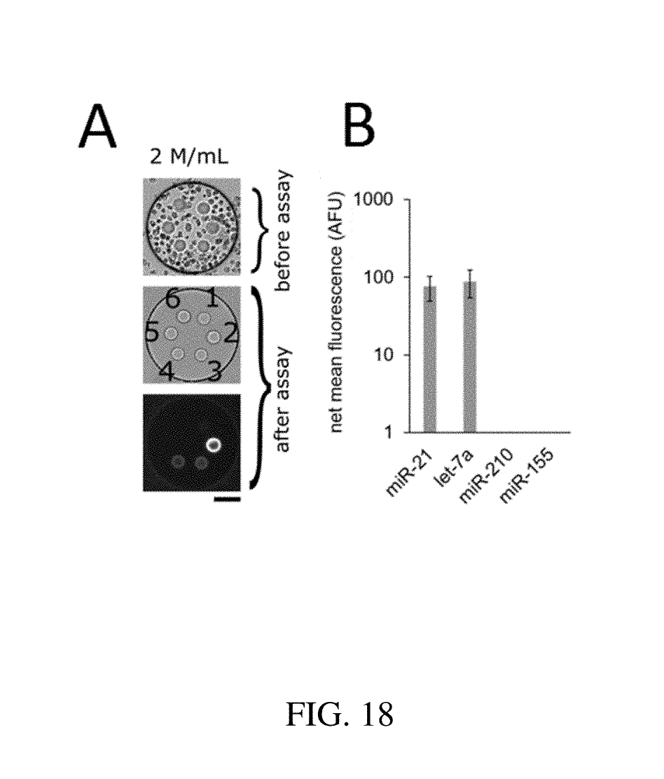

In order to demonstrate multiplex assays from unprocessed cells, we settled Calu-6 cells into wells containing six posts functionalized with probes complimentary to different miRNA targets (cel-miR-238, cel-miR-54, miR-21, miR-let-7a, miR-210, miR-155). Cells suspended at a density of 2 million cells/mL (2 M/mL) resulted in an average of 98±50 cells per well (n=16 wells) after settling for 10 min (e.g., FIG. 18A).

FIGS. 18A-B show multiplex miRNA assays from Calu-6 cells in a well array. FIG. 18A shows brightfield images after cell settling (top), and following assay (middle); and fluorescence micrograph (bottom) (at same contrast) of representative well following assay. Scale bars are 100 μm. The circular posts contained DNA probes complementary to (1) cel-miR-238, (2) cel-miR-54, (3) miR-21, (4) let-7a, (5) miR-210, and (6) miR-155, as labeled. 0.12 amol of synthetic cel-miR-54 was included in the lysis solution as a positive control and cel-miR-238 was used as a negative control. Cells were settled at a suspension density of 2 million/mL resulting in 98.2±50.4 cells per well. FIG. 18B is a plot of net mean fluorescence for the miRNA targets. Error bars indicate ±SD (n=16 wells).

0.12 amol of synthetic cel-miR-54 target was included in the lysis solution to serve as a positive control. The post with probes complementary to cel-miR-238 was used as a negative control and the mean signal from this post was subtracted from the mean signal from other posts in a given well to calculate the net mean intensity for each miRNA. The posts with probes complementary to cel-miR-54 had an average net mean intensity of 180±31 AFU (n=16 wells), which was consistent with the expected signal for the amount of miRNA mass delivered, demonstrating that the presence of the cells and lysis reagents did not interfere with the assay. Net mean positive signal was measured for miR-21 and let-7a, with miR-21 having ˜15% lower signal compared to let-7a. Signals for miR-210 and miR-155 were below the LLOD (e.g., FIG. 18B). Normalizing the resulting fluorescence signal by the number of cell per well rendered each well a biological replicate. In order to validate these results, analogous experiments were performed measuring the same miRNA targets from Calu-6 cells using a particle-based assay. Using the particle-based assay with ˜64,000 Calu-6 cells per tube, net mean positive signal was detected for miR-21 and let-7a, with miR-21 having ˜32% lower signal compared to let-7a. While miR-155 was detected using particles, its signal was ˜28× lower compared to let-7a signal, meaning it was below the LLOD of the well array assay when using ˜100 cells per well. miR-210 signal was not detected in the Calu-6 cells in either assay. Using calibration curves for miR-21 for the particle assay and the well array (e.g., FIG. 17B), the miR-21 copy number per cell in both assay formats was estimated. Comparable values of ˜2000 miR-21 copies per cell were obtained using the particle assay and ˜1000 miR-21 copies per cell using the well array assay. In the well array assay, the signal for cel-miR-54 (which served as a positive control) did not show correlation with cell number per well (R²=0.00), as expected.

c. Advantages and Improvements Over Existing Methods, Devices or Materials.

Nucleic acid sequencing from single cells is emerging as one way to study tumor heterogeneity at the gene expression level, but it has multiple challenges, including amplification artifacts, limited multiplexing, and loss of spatial information within a field of cells from the original biopsy. In situ hybridization provides spatial information, but is low-throughput, not generally multiplexible for nucleic acids and is not quantitative. Moreover, most approaches are time-consuming, costly, and clinically impractical, lacking either multiplexing, throughput, or both. Specifically for miRNA, only single-plex assays have been developed which routinely take over 6 hours and are typically not quantitative. This represents a major technical hurdle to transform single cell miRNA signatures to clinical diagnosis and stratification of human cancers. To summarize, other than the technology of the current disclosure and illustrated in this non-limiting example, there is currently no other technology that can perform high-throughput, multiplexed detection of miRNA biomarkers from tumor cells while preserving the spatial information of tumor biopsies, which are currently critical for accurate diagnosis of cancer pathology. The ability to read out miRNA levels in individual cells from a pathological specimen could add significant data points to allow better diagnostic and therapeutic accuracy for pathologists and patients.

d. Commercial Applications (Economic Potential, etc.)

The approach presented here addresses a technological gap by allowing multiplexed miRNA measurements from single cells while preserving information about spatial heterogeneity. The concept of using gel posts in microwells for cell assays introduced in the current disclosure could be extended to a number of analytes. Furthermore, using an apparatus (e.g., gel pad) to both permeabilize cells and capture their contents to collect spatially-resolved molecular data is a unique approach to interrogating tissues. The same principles of compartmentalization and reagent delivery for cell lysis and analyte extraction could be applied to other molecules such as proteins, messenger RNA, DNA, etc. The devices described in this example could be used to provide pathologists with quantitative miRNA readouts following protein or histological staining of tissues. The assay can be used not only for lung cancer, but for other cancers, neurodegenerative diseases, plus other biological and medical applications.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An apparatus comprising:
a first microwell array, wherein each microwell in the first microwell array comprises:
a first post in the microwell;
wherein the first post comprises a first probe configured to detect a first analyte.

2. The apparatus of claim 1, wherein the first post further comprises a second probe configured to detect a second analyte, wherein the second analyte differs from the first analyte.

3. The apparatus of claim 1, wherein the first post comprises a plurality of the first probe.

4. The apparatus of claim 1, wherein each microwell further comprises a second post in the microwell;
wherein the second post comprises a second probe configured to detect a second analyte;
wherein the second analyte differs from the first analyte.

5. The apparatus of claim 1, wherein the first microwell array is configured such that the contents of each microwell in the first microwell array are physically isolated from the contents of every other microwell in the microwell array.

6. The apparatus of claim 1, further comprising an enclosing structure that at least partially physically separates each microwell from an external environment.

7. The apparatus of claim 1, further comprising a substrate, a surface of which substrate is proximate a surface of the first microwell array.

8. The apparatus of claim 7, wherein the substrate comprises a second microwell array.

9. The apparatus of claim 8, wherein each microwell in the second microwell array has a smaller largest lateral dimension and/or a smaller spacing between microwells than the largest lateral dimension and/or the spacing between microwells of the microwells in the first microwell array.

10. The apparatus of claim 1, wherein the first analyte comprises a cell or molecule from a biological sample.

11. The apparatus of claim 10, wherein the biological sample is from an organism having a disease state.

12. The apparatus of claim 1, wherein the first analyte comprises a nucleic acid.

13. The apparatus of claim 12, wherein the first probe is configured to hybridize with the nucleic acid of the first analyte.

14. The apparatus of claim 1, wherein the first probe comprises a nucleic acid.

15. The apparatus of claim 12, wherein the nucleic acid of the first analyte comprises a microRNA (miRNA).

16. The apparatus of claim 12, wherein the miRNA of the first analyte comprises let-7, miR-34, miR-21, or miR-155.

17. The apparatus of claim 14, wherein the nucleic acid of the first probe comprises a deoxyribonucleic acid (DNA).

18. The apparatus of claim 14, wherein at least a portion of the nucleic acid of the first probe is complementary to at least a portion of the analyte.

19. The apparatus of claim 1, wherein the first post comprises a hydrogel.

20. The apparatus of claim 19, wherein the hydrogel comprises polyethylene glycol.

21. The apparatus of claim 1, wherein the first post comprises a polymer.

22. The apparatus of claim 21, wherein the polymer comprises polyethylene glycol.

23. The apparatus of claim 1, wherein the first post protrudes from the base of the microwell.

24. The apparatus of claim 1, wherein the first post has an aspect ratio of between or equal to 1 and 40.

25. The apparatus of claim 24, wherein the first post has an aspect ratio of between or equal to 1 and 5.

26. The apparatus of claim 1, wherein the first post has a largest lateral dimension of between or equal to 1 micron and 80 microns.

27. The apparatus of claim 26, wherein the first post has a largest lateral dimension of between or equal to 1 micron and 40 microns.

28. The apparatus of claim 1, wherein the first post has a largest lateral cross-sectional area of between or equal to 0.1 micron² and 1300 micron².

29. The apparatus of claim 28, wherein the first post has a largest lateral cross-sectional area of between or equal to 0.1 micron² and 300 micron².

30. The apparatus of claim 1, wherein the first microwell array comprises between or equal to 1 and 1000 microwells.

31. The apparatus of claim 1, wherein each microwell has a largest lateral dimension of between or equal to 5 microns and 1000 microns.

32. The apparatus of claim 31, wherein each microwell has a largest lateral dimension of between or equal to 25 microns and 400 microns.

33. The apparatus of claim 1, wherein each microwell has a depth of between or equal to 1 micron and 50 microns.

34. The apparatus of claim 33, wherein each microwell has a depth of between or equal to 30 microns and 40 microns.

35. The apparatus of claim 1, wherein each microwell is configured to contain a volume of between or equal to 0.1 nL and 99 microliters.

36. The apparatus of claim 35, wherein each microwell is configured to contain a volume of between or equal to 0.1 nL and 10 nL.

37. The apparatus of claim 1, wherein an interior surface of each microwell is cylindrical.

38. The apparatus of claim 1, wherein the interior surface of each microwell has a circular cross-section.

39. The apparatus of claim 1, wherein each microwell comprises a polymer.

40. The apparatus of claim 1, wherein each microwell comprises glass and/or quartz.

41. A method of assaying a first analyte in a biological sample, the method comprising:
exposing a biological sample to a first probe on a first post in a first microwell, wherein the first probe is configured to detect a first analyte.

42. The method of claim 41, comprising capturing the first analyte with the first probe.

43. The method of claim 42, further comprising exposing the first post to a ligation solution after capturing the analyte.

44. The method of claim 43, further comprising exposing the first post to a labeling solution after ligation.

45. The method of claim 44, further comprising measuring a fluorescence intensity of the first post after labeling.

46. The method of claim 45, wherein the fluorescence intensity is an average fluorescence intensity across the cross-sectional area of the first post.

47. The method of claim 41, comprising bringing the biological sample proximate a surface of the first microwell.

48. The method of claim 47, wherein bringing the biological sample proximate a surface of the first microwell comprises settling cells into the first microwell.

49. The method of claim 47, wherein bringing the biological sample proximate a surface of the first microwell comprises bringing a surface of a tissue sample proximate a surface of the first microwell.

50. The method of claim 41, comprising delivering an agent to the first microwell.

51. The method of claim 50, wherein the agent comprises a cell lysis agent, an extraction agent for the first analyte, and/or a capture agent for the first analyte.

52. The method of claim 50, wherein delivering an agent to the first microwell comprises:
wetting a substrate with a liquid comprising the agent; and
bringing a surface of the substrate proximate a surface of the microwell.

53. The method of claim 50, wherein delivering an agent to the first microwell comprises at least partially filling the first microwell with a liquid comprising the agent.

54. A method of assaying an analyte in a tissue sample, the method comprising:
positioning separate probe articles proximate to separate areas of a surface of the tissue sample, wherein at least some of the separate probe articles are configured to detect a first analyte.

55. The method of claim 54, comprising positioning, essentially simultaneously, the separate probe articles proximate to the separate areas of the surface of the tissue sample.

56. The method of claim 54, comprising capturing the first analyte with at least some of the separate probe articles.

57. The method of claim 54, comprising delivering an agent to the surface of the tissue sample and/or to at least some of the separate probe articles.

58. The method of claim 54, wherein at least some of the separate probe articles are posts attached to a common substrate.

59. The method of claim 58, wherein the common substrate is a microwell array.

60. The method of claim 54, wherein each of the separate probe articles have a largest lateral dimension of between or equal to 1 micron and 80 microns.

61. The method of claim 60, wherein each of the separate probe articles have a largest lateral dimension of between or equal to 1 micron and 40 microns.

62. The method of claim 54, comprising applying separate probe articles essentially simultaneously to separate areas of a surface of a tissue sample.

63. A method of delivering an agent, the method comprising:
wetting a first substrate with a liquid comprising the agent; and
positioning a surface of the first substrate proximate to a surface of a first microwell array such that the contents of each microwell in the first microwell array are physically separated from one another.

64. The method of claim 63, further comprising settling cells into the first microwell array.

65. The method of claim 63, further comprising introducing probe articles into the first microwell array.

66. The method of claim 63, wherein the agent comprises a cell lysis agent, an extraction agent for a first analyte, and/or a capture agent for a first analyte.

67. The method of claim 63, wherein positioning the surface of the first substrate proximate to the surface of the first microwell array creates sealed enclosures having a volume between or equal to 0.1 nL and 50 microliters.

68. The method of claim 63, wherein wetting comprises depositing the liquid onto the first substrate.

69. The method of claim 63, wherein the method further comprises maintaining the positioning of the first substrate relative to the first microwell array.

70. The method of claim 69, wherein the positioning is maintained using a magnet.

71. The method of claim 69, wherein the positioning is maintained using a clamp.

72. The method of claim 63, wherein the first substrate comprises a second microwell array.

73. The method of claim 72, wherein the microwells of the second microwell array have a largest lateral dimension smaller than the largest lateral dimension of the microwells of the first microwell array and/or smaller than a spacing between the microwells of the first microwell array.
